SEQUENCE LISTING (1 sequence)

SEQ ID NO: 1 - protein, 220 aa, Escherichia coli
  MUTAGEN (6)..(6): Amino acid at this position may be substituted by any of the amino acids A, L, N, Q, S, T, E, H, V, G, I or P
  MUTAGEN (26)..(26): Amino acid at this position may be substituted by any of the amino acids V, A, L, I or P
  MUTAGEN (165)..(165): Amino acid at this position may be substituted by the amino acid G
MELYLDTSDVVAVKALSRIFPLAGVTTNPSIIAAGKKPLDVVLPQLHEAMGGQGRLFAQVMATTAEGMVNDALKLRSIIADIVVKVPVTAEGLAAIKMLKAEGIPTLGTAVYGAAQGLLSALAGAEYVAPYVNRIDAQGGSGIQTVTDLHQLLKMHAPQAKVLAASFKTPRQALDCLLAGCESITLPLDVAQQMISYPAVDAAVAKFEQDWQGAFGRTSI

(19) United States
(12) Patent Application Publication
(10) Pub. No.: US 2020/0165640 A1
(43) Pub. Date: May 28, 2020

(54) FRUCTOSE-6-PHOSPHATE ALDOLASE VARIANTS FOR ALDOL CARBOLIGATIONS

(71) Applicants: CONSEJO SUPERIOR DE IVESTIGACIONES CIENTIFICAS (CSIC), MADRID (ES); TECHNICAL UNIVERSITY OF DARMSTADT, DARMSTADT (DE); SUSTAINABLE MOMENTUM, S.L., LAS PALMAS DE GRAN CANARIA (ES)

(72) Inventors: Pedro CLAPÉS SABORIT, Barcelona (ES); Jesus JOGLAR TAMARGO, Barcelona (ES); Jorge BUJONS VILAS, Barcelona (ES); Raque! ROLDAN GARCIA, Barcelona (ES); Wolf-Dieter FESSNER, Darmstadt (DE); Sebastian JUNKER, Darmstadt (DE); Pabio DOMINGUEZ DE MARIA, Las Palmas de Gran Canaria (ES)

(21) Appl. No.: 16/604,127

(22) PCT Filed: Apr. 5, 2018
(86) PCT No.: PCT/EP2018/058729

§ 371 Date: Oct. 9, 2019

(30) Foreign Application Priority Data
Apr. 10, 2017 (EP) 17382194.3

(51) Int. Cl. C12P 7/38; C12N 9/88

(52) U.S. Cl. CPC C12P 7/38; C12Y 401/02013; C12N 9/88

(57) ABSTRACT

The invention provides new and alternative fructose-6-phosphate aldolase (FSA) variants which enable the production of optically active building blocks with high chemoselectivity and stereoselectivity using aldehydes as starting material in aldol carboligation reactions, while avoiding the by-product formation and subsequent reactions.

The invention relates to the field of chemistry, specifically to aldol carboligation reactions. More particularly, the invention refers to aldolase enzyme variants that can be used as improved biocatalysts in these reactions, because they selectively produce optically active compounds with high stereo- and chemoselectivity, avoiding by-product formation.

### BACKGROUND ART

The aldol reaction is a whole class of reactions between enolates and carbonyl compounds. These are very important reactions in organic synthesis, because when the nucleophilic enolate attacks the electrophilic aldehyde or ketone, a new carbon-carbon bond is formed. Among different types of aldol reactions, aldehydes are of particular interest as donor molecules, because the product formed are aldehydes themselves and they can be subjected to further addition and lead to more complex building blocks.

The enantioselective aldol carboligation of aldehydes to afford optically active compounds is an attractive strategy to generate value. Starting materials are often readily available and inexpensive, and the obtained products are very versatile for many chemical segments and have high added value. However, despite the potential that such reactions may have, their practical use is hampered in many cases due to the intrinsic high reactivity of aldehydes, what makes these reactions prone to suffer from by-product formation, crotonizations, oligomerizations, racemization and other subsequent (and parallel) reactions. The needed purification of the desired target products from the (aqueous) complex crude mixture of by-products obtained at the end of the reaction is rather cumbersome, and in many cases virtually impossible from a practical viewpoint. Thus, these synthetic approaches cannot be used at industrial level.

An illustrative example of this problem would be the cross carboligation between chloro-ethanal and ethanal to (selectively) afford 4-chloro-3-hydroxy-butanal (3):

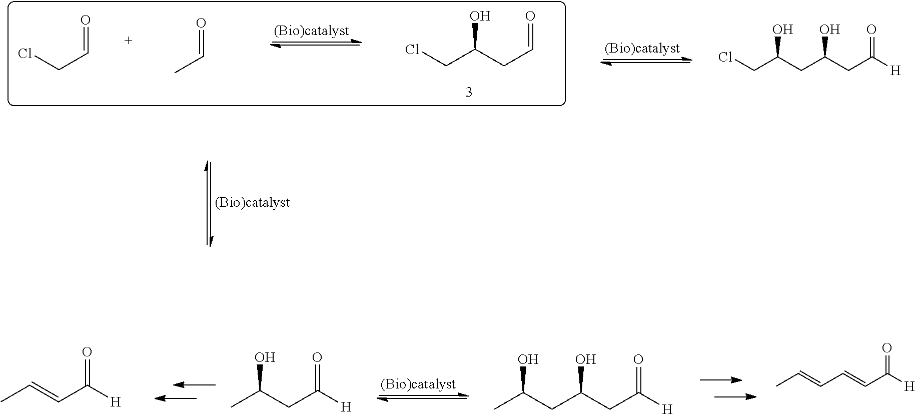

This reaction represents an example of the challenging selective cross carboligation between highly reactive aldehydes. In the square the desired (selective) process is depicted, whereas in the rest of the figure some of the possible by-products that are typically observed in crude mixtures of those reactions are shown.

In this exemplary case, compound (3) may be a useful building block for pharmaceutical and fine chemical applications, as well as for natural compounds synthesis. Yet the process is hampered by the number of by-products and subsequent (and parallel) reactions that may take place, either catalyzed or spontaneously produced. An example of this complexity and kinetics of the mixed processes can be found on literature as well (Rucigaj and Krajnc, Chem. Eng. J. 2015, 259, 11-24).

Given the importance that, for instance, 4-chloro-3-hydroxy-butanal (3) may have for synthetic purposes, several patents disclose its synthesis. Thus, CN103145540 reports on the ozonization of 5-halogenated-4-hydroxy-1-pentene compounds, leading to the desired aldehyde, which is further used via Wittig-type reactions and epoxidations, with application in the synthesis of natural products. Likewise, U.S. Pat. No. 8,742,176 discloses the use of pyrrolydine-based organocatalysis for the enantioselective addition of ethanal to chloroethanal, again with subsequent Wittig-type condensation and epoxidation. With regard to biocatalysis, EP1734129 focuses on the enzyme 2-deoxyribose-5-phosphate aldolase (DERA) for the production of (3), as well as on the further addition of another ethanal molecule to (3), to afford the statin-chain precursor. In that patent, it is mentioned that DERA from Pyrobaculum aerophilum is selective for the mono-addition of two aldehydes (namely choroethanal and ethanal, among other examples). However, a careful reading of that patent shows that such enzyme displays not-complete selectivity for the single addition, but the double addition is still present (example 7, page 12 of said patent). Thus, a chromatographic separation of the single and the double addition adducts is still needed at the end of the reaction.

Aldol reactions have been extensively studied from different perspectives including the development of new catalysts for stereocontrolled synthesis. Catalysts that have been developed for asymmetric aldol reactions include small organic molecules, transition metal coordination complexes with chiral ligands and aldolases. The synthetic utility of aldol reactions would be greatly enhanced if the formation of enolate anion and the attack of carbonyl group could be chemically selective in a cross aldol reaction to avoid the formation of complex products mixture. Since enzymes exert high level of selectivity that is not usually possible in simple chemical reaction in the laboratory, aldolases have emerged as an environmentally friendly alternative with great application potentials in the synthesis of rare sugars or sugar derivatives such as statins, iminocyclitols, epothilones and sialic acids.

Aldolases (EC 4.1.2.X) are lyases that typically catalyze the stereoselective addition of a ketone or aldehyde donor to an aldehyde acceptor. Up until now, there are more than 30 known aldolases present in most, if not all, organisms. Aldolases are nature's own catalysts for one of the most fundamental reactions in organic chemistry: the formation of new carbon-carbon bonds. In biological systems, aldol formation and cleavage reactions play central roles in sugar metabolism. In organic synthesis, as previously mentioned, aldolases attract great attention as environmentally friendly alternative for the synthesis of polyhydroxylated compounds in stereocontrolled manner.

Thus, approaches for designing aldolases with improved donor selectivity have been developed. For instance, fructose-6-phosphate aldolase (FSA) variants from E. coli with single, double or triple amino acid substitutions have been described in Anna Szekrenyi, et al., 2014, Chemistry A European Journal, Engineering the Donor Selectivity of D-Fructose-6-Phosphate Aldolase for Biocatalytic Asymmetric Cross-Aldol Additions of Glycolaldehyde. Specifically in this prior art document, the following E. coli FSA variants were disclosed: L107Y, A129G/A165G, A129S/A165G, A129T/A165G, A129G, A129V, A129T, A129S, L107Y/A129G, L107H/A129G, L107F/A129G, L107H, L107Y/A129G/A165G, L107H/A129G/A165G and L107F. The selectivity of these variants towards donor substrates, such as glycolaldehyde and dihydroxyacetone, was assessed. Other FSA variants, such as A165G, A129S and A165G/A129S have been found to be selective for additions of hydroxypropanone and dihydroxypropanone (Gutierrez, et al., Chem. Commun. 2011, 47 (20), 5762-5764). However, whereas all these variants are selective for hydroxymethyl ketones as nucleophile components they are inactive for those without the hydroxymethyl functionality. Therefore, no FSA variants have been described to be active toward nucleophiles without hydroxym ethyl functionality and non-phosphorylated electrophiles.

Furthermore, E. coli FSA variants with increased catalytic activity for aldol reaction with phenylacetaldehyde and hydroxyacetone as substrates have been described in Huan Ma. 2015. Digital Comprehensive Summaries of Uppsala Dissertations from the Faculty of Science and Technology 1318. 67. Particularly, these variants were obtained by mutagenesis around the substrate binding site near catalytic Lys85, Arg134 and Ser166. However, the same drawbacks as those indicated above for the FSA variants of the state of the art can be applied for these variants.

As can be seen, based on the synthetic potential of compounds such as 4-chloro-3-hydroxy-butanal, (3), and others—as well as their expected high market prices—, there is a need for selective (bio)catalysts that may synthetize exclusively the desired target, while avoiding the by-product formation and subsequent reactions, as well as further downstream processing units. Having those (bio)catalysts in hands, the production of highly valuable (optically active) building blocks with high chemoselectivity and stereoselectivity would facilitate the set-up of econom ical approaches using lyases (aldolases) and highly reactive (inexpensive) aldehydes as starting materials.

To date, no such high stereoselective-(bio)catalysts exist in the literature for the intended reactions, and only DERA from Pyrobaculum aerophilum seems to show some selectivity for (3) compared to subsequent additions of aldehydes to the formed product (3). Therefore, it is industrially very important to develop (bio)catalysts for producing predominantly the desired product, with high chemo- and stereoselectivity, in aldol carboligation reactions from aldehydes.

### DESCRIPTION OF THE INVENTION

This invention is related to enzymatic processes based on E. coli fructose-6-phosphate aldolase (FSA) variants catalysis which provides chiral synthons and other products with high potential interest for synthetic processes with excellent chemoselectivity and stereoselectivity under environmentally friendly conditions.

Particularly, the invention provides new and alternative FSA variants (mutants) which enable the production of highly valuable (optically active) building blocks with high chemoselectivity and stereoselectivity using aldehydes as starting material in aldol carboligation reactions, while avoiding the by-product formation and subsequent reactions.

The present invention discloses several FSA variants that are suitable and advantageous as enzymatic biocatalysts for the chemoselective single C—C bond formation of different building blocks. When using these FSA variants of the invention, the by-products expected in these aldol carboligation reactions (see figure above) are not found, since these variants are fully selective for the single addition of the aldehydes used as substrates. Therefore, FSA variants of the invention are highly selective and only two substrates are reacting in their presence to produce a single condensed product, the reaction stops at that desired compound, i. e. only the desired product is formed. This approach allows thus the use of highly reactive aldehydes as substrates.

The invention provides, therefore, an enantioselective or diastereoselective biocatalytic aldol reaction (C—C bond formation) using aldehydes and ketones as substrates, leading exclusively to the single, chemo- and stereoselective carboligation of two substrates—which may be similar or different between them—with no formation of by-products or further consecutive reactions adding more aldehydes. Selectivity in the single addition is always >99.99%, as the FSA aldolase variants of the invention, when used as biocatalysts, show exclusive selectivity for the single aldol addition of two substrates.

Therefore, the designed FSA variants of the invention have remarkable high selectivity in so challenging reactions, producing compounds with high potential interest for synthetic processes, whereas the FSA wild-types are not able to catalyze these reactions at all.

No other (bio)catalysts able to perform these reactions are known, being so selective, in which only two substrates are reacting to produce a single condensed product and reaction stops at that desired compound. The only example is the above-reported DERA, in which some (incomplete) selectivity for the single addition is noted. In the present invention, as a remarkable example for the FSA variants, in the case of compound 4-chloro-3-hydroxy-butanal (3), very promising productivities (space-time-yields of 10-12 g L⁻¹d⁻¹) have been achieved. This suggests that after careful fine-tuning and optimization, robust and fully selective biocatalysts have been obtained in the present invention. Moreover, these aldol carboligation reactions have been studied in the presence of the E. coli FSA wild type or each of the following FSA variants: A165G, L107A, L163A, A129G, L107Y/A129G, A129V, A129T, A129S, L107Y/A129G, L107H/A129G, L107F/A129G, L107H, L107Y/A129G/A165G, L107 H/A129G/A165G, L107F, L107Y/A129G/A165G/S166G, but reactions did not take place or proceeded with low chemo- and stereoselectivity.

Moreover, these FSA variants disclosed in the present invention are selective for both hydroxym ethyl ketones as nucleophile components and for those without the hydroxym ethyl functionality. No FSA variants had been described up to date to be active toward nucleophiles without hydroxymethyl funcionality and non-phosphorylated electrophiles.

As explained above, aldol reactions are very useful but when highly reactive aldehydes are employed their practical use is hampered. Work-up of a complex mixture is virtually uneconomic. However, with the new FSA-variants-based approach presented herein downstream is significantly simplified, as only the desired product is formed. Moreover, given the mild conditions of the processes, it is possible to perform enzymatic multi-step processes to further derivatization of the obtained compounds.

Thus, the present invention solves the aforementioned problems by providing FSA aldolase variants that selectively produce the desired product as well as a process for producing optically active compounds with high chemo- and stereos electivity.

Specifically, the inventors found that mutagenesis (preferably, amino acid substitutions) at a particular position, namely position 6, of the SEQ ID NO: 1 (which is the wild type amino acid sequence of the FSA from Escherichia coli) gives FSA variants that lead to a high chemo- and stereoselectivity when used as biocatalysts in aldol carboligation reactions. This results in FSA variants producing predominantly the desired optically active product with high selectivity, preferably with improved selectivity compared to the wild type sequence of SEQ ID NO: 1. Therefore, the inventors propose this position 6 in the amino acid sequence of the wild type FSA enzyme from E. coli for the design of FSA enzyme variants that lead to a high chemo- and stereoselectivity in aldol carboligation reactions. This particular amino acid residue located at position 6 of the FSA sequence SEQ ID NO: 1 has not been associated before to the substrate specificity or the stereoselectivity of the enzyme.

Thus, a first aspect of the invention refers to a fructose-6-phosphate aldolase variant comprising:
a. an amino acid sequence having at least 80%, preferably at least 85%, 90%, 91%, 92%, 93%, 94%, 95%, 96%, 97%, 98% or 99%, more preferably at least 90%, more preferably at least 95%, even more preferably at least 99%, sequence identity with SEQ ID NO: 1, and
b. an amino acid substitution at position 6, corresponding to positions 1 to 220 of SEQ ID NO: 1.

Hereinafter, this variant will be also referred to as “variant of the invention” or “FSA variant of the invention.”

The FSA variants encompassed within the present invention lead to a high enantioselectivity or diastereoselectivity when used as a biocatalyst in a reaction of aldol carboligation, more preferably to an enhanced, increased or improved enantioselectivity or diastereoselectivity compared to the FSA of SEQ ID NO: 1.

The term “enantioselectivity” means the selectivity of a reaction towards one of a pair of enantiomers. The property of “enantioselectivity” may be assessed by any of the methods known in the art for the determination of enantioselectivity. For instance, the enantioselectivity may be determined by measuring the enantiomeric excess after the aldol carboligation reaction performed in the presence of the FSA variant of the invention. The term “enantiomeric excess” (ee) refers to the difference between the amounts of each of the enantiomers present in a mixture, relative to the total amount of the compound in the mixture expressed as percentage (×100%). For an excess of the R-enantiomer, the enantiomeric excess can be calculated by the following formula:

ee %=([R]−[S])/([R]+[S])×100

For an excess of the S-enantiomer, the enantiomeric excess can be calculated by the following formula:

ee %([S]−[R])/([R]+[S])×100

In the above formulas, [R] and [S] are the respective molar fractions of the enantiomers in a mixture such that [R]+[S]=1.

The amounts of each of the enantiomers present in a mixture may be measured, for instance but without limitation, by HPLC, Chiral GC, and the like.

The term “diastereoselectivity” means the selectivity of a reaction towards one of a pair of diastereoisomers. The property of “diastereoselectivity” may be assessed by any of the methods known in the art for the determination of diastereoselectivity. For instance, the diastereoselectivity may be determined by measuring the diastereomeric ratio after the aldol carboligation reaction performed in the presence of the FSA variant of the invention. The term “diastereomeric ratio” (dr) refers to the ratio of the percentage of one diastereoisomer in a mixture to that of the other.

The amounts of each of the diastereoisomers present in a mixture may be measured, for instance but without limitation, by NMR.

According to the present invention, the enantioselectivity or diastereoselectivity of a reaction in which the FSA variant of the invention is used as a biocatalyst is “high” when the enantiomeric excess is, preferably, 95% or higher, more preferably 99% or higher, or the diastereomeric ratio is, preferably, 95 or higher, more preferably 99 or higher.

“Fructose-6-phosphate aldolase” or “FSA” is the enzyme classified as EC 4.1.2.X and the most recently discovered member of the Class I aldolase family. It exists as isoenzymes: FSAA and FSAB coded by mipB and talC genes in E. coli, respectively. The two isoenzymes have similar substrate profiles but FSAB shows lower catalytic activity and therm ostability. FSA is the first reported enzyme capable of catalyzing the cleavage of fructose-6-phosphate to generate DHA and G3P. The amino acid sequence of wild type FSA referred to in the present invention is SEQ ID NO: 1.

The term “variant”, as used herein, relates to a FSA enzyme that comprises at least one mutation, preferably at least one amino acid substitution, compared to the E. coli wild type FSA (SEQ ID NO: 1) at the points(s) described herein within its amino acid sequence and, therefore, has a different sequence to that of the natural or wild-type enzyme of SEQ ID NO: 1. As used herein, the expression “FSA variant” means preferably a polypeptide comprising the amino acid substitutions described herein and having fructose-6-phosphate aldolase activity, which have been produced by chemical synthesis or recombinantly by an organism that expresses a nucleotide sequence that encodes the FSA wild type, preferably of SEQ ID NO: 1, modified in the sense described herein. Said modified sequence is obtained by means of human intervention by modifying the nucleotide sequence that encodes the native or wild type FSA (preferably the wild type FSA of SEQ ID NO: 1). The term “modification” means any chemical modification of the amino acid or nucleic acid sequence of native or wild type FSA, preferably of SEQ ID NO: 1.

The term “identity” or “homology”, as used herein, in the context of describing two or more polypeptide sequences, relates to a specified percentage of coincidences of amino acid residues at the positions from an alignment of two amino acid sequences. Sequence alignment methods for comparison are well known in the state of the art. The degree of identity can be determined using the Clustal method (Higgins, 1989, CABIOS 5: 151-153), the Wilbur-Lipman method (Wilbur y Lipman, 1983, Proceedings of the National Academy of Science USA 80: 726-730), the GAG program, including GAP (Devereux et al. 1984, Nucleic Acids Research 12: 287 Genetics Computer Group University of Wisconsin, Madison, 25 (WI)); BLAST or BLASTN, EMBOSS Needle and FASTA (Altschul et al. 1999, J. Mol. Biol. 215: 403-410). Additionally, the Smith-Waterman algorithm can also be used for the purpose of determining the degree of identity between two sequences.

Preferably, the degree of identity is determined using BLAST with the default parameters. This software is publicly available at the National Center for Biotechnology Information (NCBI).

The FSA variants of the invention, apart from the amino acid substitutions at the specific positions indicated herein, can exhibit other limited changes in their amino acid sequences. These changes make it possible to maintain the fructose-6-phosphate aldolase activity of the FSA variants of the invention and maintain the property of leading to a high chemo- and stereoselectivity in aldol carboligation reactions. Said changes may be substitutions, deletions or additions. Substitutions are by conserved amino acids, which are amino acids with lateral strands and properties similar with respect to, for example, hydrophobic or aromatic properties. These substitutions include, but are not limited to, substitutions between Glu and Asp, Lys and Arg, Asn and Gln, Ser and Thr, and/or between the amino acids included in the following list: Ala, Leu, Val and Ile. The changes referred to in this paragraph do not lead to relevant modifications in the essential characteristics or properties of the variants of the invention.

The individual amino acids in an amino acid sequence are represented herein as XN, where X is the amino acid in the sequence (designated by means of the one letter code universally accepted in amino acid nomenclature) and N is the position in the sequence. The amino acid substitutions are represented herein as X₁NX₂, where X₁ is the amino acid in the sequence of the non-mutated enzyme, X₂ is the new amino acid in the sequence of the mutated enzyme (variant) and N is the position in the amino acid sequence in relation to the positions of SEQ ID NO: 1.

In a preferred embodiment of the variant of the invention, the amino acid substitution at position 6 is by an amino acid selected from the list consisting of: A, L, N, Q, S, T, E, H, V, G, I or P, more preferably by H, N, Q, L, T, E or A. More preferably, the amino acid substitution is selected from the list consisting of: D6A, D6L, D6N, D6Q, D6S, D6T, D6E, D6H, D6V, D6G, D6I or D6P, preferably D6H, D6N, D6Q, D6L, D6T, D6E or D6A.

In a more preferred embodiment, the variant of the invention comprises, even more preferably consists of, the amino acid sequence SEQ ID NO: 1 in which the D at position 6 (D6) has been substituted by an amino acid selected from the list consisting of: A, L, N, Q, S, T, E, H, V, G, I or P, preferably by H, N, Q, L, T, E or A.

In an even more preferred embodiment, the variant of the invention comprises the amino acid substitution D6N or D6H. In a particular embodiment of the present invention, the variant comprises, even more preferably consists of, the amino acid sequence SEQ ID NO: 1 in which the D at position 6 (D6) has been substituted by N or H.

In another preferred embodiment, the variant of the invention further comprises an amino acid substitution at position 26, corresponding to positions 1 to 220 of SEQ ID NO: 1. More preferably, this amino acid substitution at position 26 is by an amino acid selected from the list consisting of: V, A, L, I or P, more preferably by L or I. Even more preferably, this amino acid substitution is selected from the list consisting of: T26V, T26A, T26L, T26I or T26P, preferably T26L or T26I.

In a particular embodiment, the variant of the invention comprises the amino acid substitutions D6A and T26L or D6A and T26I.

In another preferred embodiment, the variant of the invention further comprises an amino acid substitution at position 165, corresponding to positions 1 to 220 of SEQ ID NO: 1, by the amino acid G. More preferably, this amino acid substitution is A165G.

In another particular embodiment, the variant of the invention comprises the amino acid substitutions D6E and A165G or D6L and A165G.

In another particular embodiment, the variant of the invention comprises amino acid substitutions at positions 6, 26 and 165, corresponding to positions 1 to 220 of SEQ ID NO: 1, more preferably by any of the amino acids indicated above for each position.

Amino acid substitutions described herein introduced in the wild-type polypeptide sequence of FSA can be obtained using genetic engineering techniques or recombinant DNA, such as for example by mutating the encoding sequence of the wild type FSA enzyme by means of directed mutagenesis, or they can be obtained by means of chemical synthesis of the nucleotide sequence which codes for the sequence of the variant of the invention that carries said amino acid substitutions.

Therefore, the FSA variant of the invention can be synthesised, for example, but without limitations, in vitro. For example, by means of the synthesis of solid-phase peptides or recombinant DNA approaches. The variant of the invention can be produced in a recombinant manner, including its production as a mature peptide or as a pre-protein that includes a signal peptide.

Another aspect of the invention refers to an isolated nucleic acid sequence encoding the variant of the invention, hereinafter “nucleotide sequence of the invention”.

Due to the degeneration of the genetic code, various nucleotide sequences can encode the same amino acid sequence.

In accordance with the present invention, an isolated “nucleic acid molecule”, “nucleotide sequence”, “nucleic acid sequence” or “polynucleotide” is a nucleic acid molecule (polynucleotide) that has been eliminated from its natural medium (i.e. it has been subjected to human manipulation) and can include DNA, RNA or DNA or RNA derivatives, including cDNA. The nucleotide sequence of the present invention may or may not be chemically or biochemically modified and can be artificially obtained by means of cloning and selection methods or by means of sequencing.

The nucleic acid sequence of the invention can encode the mature polypeptide or a pre-protein consisting of a signal peptide linked to the mature enzyme that must subsequently be processed.

The nucleotide sequence of the present invention may also comprise other elements, such as introns, non-encoding sequences at 3′ and/or 5′ ends, ribosome binding sites, restriction sites, etc. This nucleotide sequence can also include encoding sequences for additional amino acids that are useful for the purification or stability of the encoded peptide.

The nucleic acid sequence of the invention can be included in a genetic construct, preferably in an expression vector. Said genetic construct may also comprise one or more gene expression-regulating sequences, such as promoters, enhancers, regulators, terminators, etc.

Therefore, another aspect of the invention refers to a gene construct comprising the nucleic acid sequence of the invention, hereinafter “gene construct of the invention”. In a preferred embodiment, said gene construct is an expression vector.

The expression “gene construct”, “genetic construct” or “nucleic acid construct” as used herein relates to a functional unit required to transfer, and preferably to express, a gene of interest, herein the nucleotide sequence of the invention as described, in a host cell. This gene construct preferably also comprises regulating or control sequences including, for example, a promoter, an enhancer, a terminator, etc. operably linked to the sequence that encodes the protein. It refers to a mono or double-stranded nucleic acid molecule, which is isolated from a natural gene or that is modified to contain nucleic acid segments in such a manner that they would otherwise not exist in nature. The expression “nucleic acid construct” is synonymous to the expression “expression cassette”, when the construct of nucleic acid contains the control sequences required for the expression of the encoding sequence. Preferably, the gene construct of the invention further comprises a promoter operably linked to the nucleic acid sequence of the invention. The promoter may be, but without limitations, a constitutive or inducible promoter. As a promoter for expressing the nucleic acid sequence of the invention, a promoter which has usually been used in production of heterologous proteins in Escherichia coli may be used, for example, powerful promoters such as T7 promoter, trp promoter, lac promoter, tac promoter, PL promoter, can be included in the construct.

Preferably, the gene construct of the invention further comprises a terminator. Examples of such a terminator include, but without limitations, T7 terminator, fd phage terminator, T4 terminator, tetracyclin resistant gene terminator, Escherichia coli trpA gene terminator, and the like.

The term “expression vector”, also known as “expression construct” or “plasmid”, relates to a DNA molecule, linear or circular, that comprises the nucleic acid sequence of the invention operably linked to additional segments that provide the transcription of the encoded peptide. Generally, a plasmid is used to introduce a specific gene in a target cell. Once the expression vector is in the interior of the cell, the protein encoded by the gene is preferably produced by means of the ribosome complexes of the cellular transcription and translation machinery. The plasmid is often subject to engineering to contain regulating sequences that act as enhancing and promoter regions that lead to an efficient transcription of the gene carried on the expression vector. The objective of a well-designed expression vector is the production of large amounts of stable messenger RNA and, therefore, of proteins.

Expression vectors are basic tools for biotechnology and for the production of proteins, such as enzymes. The expression vector of the invention is introduced in a host cell such that the vector remains as a chromosome constituent or as an extra-chromosome self-replicating vector.

The term “expression” relates to the process whereby a polypeptide is synthesised from a polynucleotide. The term includes the transcription of the polynucleotide in a messenger RNA (mRNA) and the translation of said m RNA into a protein or polypeptide.

Examples of expression vectors are, but without limitations, phages, cosmids, phagemids, yeast artificial chromosomes (YAC), bacterial artificial chromosomes (BAC), human artificial chromosomes (HAC) or viral vectors, such as adenovirus, retrovirus or lentivirus.

The appropriate expression vectors for the insertion of the polynucleotide of the invention are, preferably, plasm ids used for the protein expression in prokaryotes such as, by way of example: pUC18, pUC19, Bluescript and its derivatives, mp18, mp19, pBR322, pMB9, Co1E1, pCR1, RP4, pET plasm ids, phages and “launcher” vectors, such as pSA3 and pAT28; yeast expression vectors such as the 2-micron plasmid of Saccharomyces cerevisiae, integration plasmids, YEP vectors, centromere plasmids and similar; expression vectors in insect cells such as the vectors of the pAC and pVL series; expression vectors in plant cells such as piBi, pEarleyGate, PAVA, pCAMBIA, PGSA, PGWB, PMDC, PMY, pore series and similar, and other protein expression plasmids used in eukaryote cells, including baculovirus adequate for cell transfection.

In order to select the transformants comprising the gene construct of the invention, said gene construct preferably comprises a marker, such as an ampicillin resistant gene. Such plasm ids are commercially available and have powerful promoters (pUC series (Takara S buzo), pPROK series (Clontech), pKK233-2 (Clontech), etc.). Alternatively, the marker may be other drug resistant gene such as kanamycin, neomycin or chloramphenicol resistant gene, or a fluorescence gene, such as luciferase, GFP, mCherry, and the like. Or the marker may be an auxotrophic genetic marker.

The variant of the invention can be prepared using any known means in the state of the art, such as the modification of a DNA sequence that encodes the FSA enzyme of SEQ ID NO: 1, transformation of the modified DNA sequence in an adequate host cell and the expression of the modified DNA sequence to form the mutant or variant enzyme.

Therefore, another aspect of the invention relates to a host cell comprising the nucleic acid sequence of the invention or the gene construct of the invention, hereinafter “host cell of the invention”.

The term “host cell”, as used in this description, relates to any prokaryote or eukaryote cell that is the recipient of an expression vector, cloning vector or any other exogenous nucleic acid molecule. Therefore, the term includes any cultivable cell that may be modified through the introduction of a nucleic acid not contained naturally therein. Preferably, a host cell is that in which the polynucleotide of the invention may be expressed, giving rise to a stable polypeptide, post-translationally modified and located in the appropriate subcellular compartment. The election of an appropriate host cell may also be influenced by the election of the detection signal. For example, the use of constructs with reporter genes (for example, lacZ, luciferase, thymidine kinase or green fluorescent protein “GFP”) can provide a signal selectable through the activation or inhibition of the transcription of the gene of interest in response to a transcription-regulating protein. In order to achieve optimum selection or screening, the phenotype of the host cell must be considered.

The host cell of the invention may be, but without limitations, a bacterial cell, an actinomycetes cell, a yeast cell, a fungal cell, a plant cell, or an animal cell. Bacterial cells suitable for the transformation with the nucleic acid sequence of the invention or the gene construct of the invention are, for instance, bacteria of Escherichia, Pseudomonas, Corynebacterium, Bacillus, and the like, and Escherichia coli is preferably used because there are a large number of findings on the techniques for mass production of proteins using it.

The host cell of the invention may be cultivated for the production of the FSA variant of the invention. A host cell culture relates to the process of in vitro maintaining and growing the host cells. Cell cultures need controlled conditions of temperature, pH, proportion of gases (oxygen and carbon dioxide), etc., in addition to the presence of the adequate nutrients to permit cellular viability and division. Cell cultures can be carried out in solid substrates, such as agar, or in a liquid medium which enables the cultivation of large amounts of cells in suspension.

The culture of the host cell of the invention may be conducted in a culture medium usually employed in the art, for instance, a medium containing a carbon source, nitrogen source, inorganic salts, trace metal salts, vitamins, etc.

Thus, another aspect of the present invention refers to the use of the host cell of the invention for producing the variant of the invention.

The host cell of the invention is therefore cultured in an appropriate culture medium, and the FSA variant of the invention is accumulated in the any one of medium and cells or both.

It is possible to increase the expression of the recombinant gene in the host cell by adding to the culture medium a proper amount of an inducer responding to a promoter integrated in the gene construct of the invention. For example, when the gene construct is constructed with a lac promoter to which an objective gene is linked at the downstream, it is possible to add properly IPTG or alternatively it is also possible to add properly galactose.

Following cultivation, the host cells may be recovered by, for example, centrifugation. Since the majority of the FSA enzyme is present in the cells, the variant may be solubilized by disrupting or lysing the host cells. Ultrasonic disrupting, French press disrupting or glass bead disrupting may be used to disrupt the host cells. In the case of lysing the cells, a method that uses lysozyme, peptidase, or a suitable combination thereof may be adopted.

Afterwards, the variant of the invention can be purified. The term “purify”, as used in the description, relates to the isolation of the variant of the invention and to its concentration, disregarding the other polypeptides present in the culture medium. The isolation or purification of the FSA variant of the invention can be carried out, for instance, using differential solubility techniques, chromatography, electrophoresis or isoelectric focusing. Chromatography techniques can be based on molecular weight, ion charge (based on the ionisation state of the amino acids under working conditions), the affinity of the protein for certain matrixes or chromatographic columns, or by means of purification tags, and can be carried out for example on a column, on paper or on a plate. The isolation or purification of the enzyme variant can be carried out, for example, by means of precipitation with ammonium sulphate, gel filtration chromatography, ion exchange chrom atography, hydrophobic chrom atography, hydroxyapatite chromatography, fast protein liquid chromatography (FPLC) or high performance liquid chromatography (HPLC), using automated systems that significantly reduce purification time and increase purification efficiency.

When the FSA variant of the invention derived from the host cell of the invention is purified, although the variant is purified by using an enzyme solubilizing solution for the starting material, if undisrupted or unlysed residue remains, re-centrifuging the solubilization solution and removing any residue that precipitates is advantageous to purification.

Another aspect of the invention refers to the use of the variant of the invention or the host cell of the invention as a biocatalyst for aldol carboligation reactions (i. e. for stereoselective C—C bond formation).

The variant of the invention may be added to the reaction medium directly or as a microorganism containing (expressing) it, as far as it catalyzes the aldol carboligation reaction. Thus, the variant of the invention may be added to the reaction, for example, as a free-enzyme, as an immobilized enzyme, as a whole cell expressing it or as an immobilized whole cell expressing it.

The term “immobilized” means that the variant of the invention or the host cell of the invention is coupled to surface, i.e. it is on a surface (directly linked or linked by means of any linker molecule) or the surface is coated with it. Said surface may be, for instance, a solid or semi-solid support, a matrix, a plate, a particle, preferably a nanoparticle, a gel, a biopolymer, beans or the like.

The additive comprising the variant of the invention to be added to the reaction medium may include the culture medium (the medium from which the cells have been removed), cells (including both of cultured cells and washed cells), cells processed by crushing or lysis, a composition comprising an active FSA variant of the invention derived by purification from the above-mentioned culture medium and/or cells (crude enzyme solution, purified enzyme), and the like.

For example, when the aldol carboligation reaction is produced with a whole host cell producing the variant of the invention, the substrates of the aldol reaction may be added directly to the culture broth during the incubation of the host cell. Host cells separated from the culture broth, washed cells, or cells processed by crushing or lysis may be added to the reaction mixture, or alternatively the FSA variant of the invention is recovered from the processed cells and may be added as a crude enzyme solution or purified enzyme to the reaction mixture. That is, any form of fraction comprising the variant of the invention may be used in an aldol carboligation reaction according to the invention.

In a preferred embodiment, the aldol carboligation reaction takes place between two aldehydes or between an aldehyde and a ketone.

More preferably, the aldehydes are selected from the list consisting of: 2-chloroethanal (or chloroacetaldehyde), formaldehyde, benzyloxyethanal, propanal, pentanal, isopentanal, ethanal (or acetaldehyde), butanal, an hydroxyaldehyde or a N-Cbz-am inoaldehyde. Exam pies of hydroxyaldehyde are 3-hydroxypropanal, R-3-hydroxybutanal or S-3-hydroxybutanal. Examples of N-Cbz-aminoaldehyde are N-Cbz-3-am inopropanal or N-Cbz-glycinal.

In another preferred embodiment, the ketone is selected from the list consisting of: propanone, butanone, cyclobutanone, cyclopentanone or 3-pentanone.

Another aspect of the invention refers to a method for aldol carboligation, hereinafter “the method of the invention”, comprising the following steps:
a. reacting two aldehydes or an aldehyde and a ketone in the presence of the variant of the invention or the host cell of the invention, and
b. recovering the product of the reaction performed in step (a).

In a preferred embodiment of the method of the invention, the reaction in step (a) takes place in an aqueous buffered solution at 25° C. or less.

In another preferred embodiment of the method of the invention, the product of the reaction performed in step (a) is recovered in step (b) by means of one of the following methods: column chromatography on silica or on hydrophobic polymeric support, or on activated charcoal or on a mixture of diatomaceous earth and activated charcoal or using preparative normal or reverse phase HPLC or liquid-liquid extraction, or any combination thereof.

More preferably, the aldehydes in the method of the invention are selected from the list consisting of: 2-chloroethanal (or chloroacetaldehyde), formaldehyde, benzyloxyethanal, propanal, pentanal, isopentanal, ethanal (or acetaldehyde), butanal, an hydroxyaldehyde or a N-Cbz-aminoaldehyde. Examples of hydroxyaldehyde are 3-hydroxypropanal, R-3-hydroxybutanal or S-3-hydroxybutanal. Examples of N-Cbz-aminoaldehyde are N-Cbz-3-aminopropanal or N-Cbz-glycinal.

In another preferred embodiment, the ketone in the method of the invention is selected from the list consisting of: propanone, butanone, cyclobutanone, cyclopentanone or 3-pentanone.

In a particular embodiment of the present invention, the aldehyde is 3-hydroxypropanal, the ketone is propanone and the variant of the invention comprises the amino acid substitution D6H.

In another particular embodiment, the aldehyde is S-3-hydroxybutanal, the ketone is propanone and the variant of the invention comprises the amino acid substitution D6H.

In another particular embodiment, the aldehyde is R-3-hydroxybutanal, the ketone is propanone and the variant of the invention comprises the amino acid substitution D6H.

In another particular embodiment, the aldehyde is benzyloxyethanal, the ketone is propanone and the variant of the invention comprises the amino acid substitution D6Q.

In another particular embodiment, the aldehyde is S-3-hydroxybutanal, the ketone is cyclobutanone and the variant of the invention comprises the amino acid substitution D6H.

In another particular embodiment, the aldehyde is S-3-hydroxybutanal, the ketone is cyclopentanone and the variant of the invention comprises the amino acid substitution D6H.

In another particular embodiment, the aldehyde is benzyloxyethanal, the ketone is cyclobutanone and the variant of the invention comprises the amino acid substitution D6Q.

In another particular embodiment, the aldehyde is N-Cbz-3-aminopropanal, the ketone is propanone and the variant of the invention comprises the amino acid substitution D6N.

In another particular embodiment, the aldehyde is N-Cbz-glycinal, the ketone is propanone and the variant of the invention comprises the amino acid substitution D6T.

In another particular embodiment, the aldehyde is N-Cbz-3-aminopropanal, the ketone is butanone and the variant of the invention comprises the amino acid substitution D6E.

In another particular embodiment, the aldehyde is N-Cbz-glycinal, the ketone is butanone and the variant of the invention comprises the amino acid substitution D6T.

In another particular embodiment, the aldehyde is N-Cbz-3-aminopropanal, the ketone is 3-pentanone and the variant of the invention comprises the amino acid substitution D6E.

In another particular embodiment, the aldehyde is N-Cbz-glycinal, the ketone is 3-pentanone and the variant of the invention comprises the amino acid substitution D6T.

In another particular embodiment, the aldehyde is N-Cbz-3-aminopropanal, the ketone is cyclobutanone and the variant of the invention comprises the amino acid substitution D6L.

In another particular embodiment, the aldehyde is N-Cbz-3-aminopropanal, the ketone is cyclopentanone and the variant of the invention comprises the amino acid substitution D6L.

In another particular embodiment, the aldehyde is N-Cbz-glycinal, the ketone is cyclopentanone and the variant of the invention comprises the amino acid substitution D6Q.

Variants of the invention are fully selective for the single addition (selective mono-addition) of two aldehydes (more than 99.99% selectivity in the single addition).

Thus, in another particular embodiment, the aldol carboligation reaction takes place between two aldehydes, wherein one of the aldehydes is ethanal, the other aldehyde is 3-hydroxypropanal, and the variant of the invention comprises the amino acid substitutions D6L and A165G.

In another particular embodiment, the aldol carboligation reaction takes place between two aldehydes, wherein one of the aldehydes is ethanal, the other aldehyde is S-3-hydroxybutanal, and the variant of the invention comprises the amino acid substitution D6H.

In another particular embodiment, the aldol carboligation reaction takes place between two aldehydes, wherein one of the aldehydes is ethanal, the other aldehyde is R-3-hydroxybutanal, and the variant of the invention comprises the amino acid substitution D6H.

In another particular embodiment, the aldol carboligation reaction takes place between two aldehydes, wherein one of the aldehydes is ethanal, the other aldehyde is N-Cbz-3-am inopropanal, and the variant of the invention comprises the amino acid substitutions D6E and A165G.

In another particular embodiment, the aldol carboligation reaction takes place between two aldehydes, wherein one of the aldehydes is formaldehyde, the other aldehyde is propanal, and the variant of the invention comprises the amino acid substitution D6Q.

In another particular embodiment, the aldol carboligation reaction takes place between two aldehydes, wherein one of the aldehydes is ethanal, the other aldehyde is 2-chloroethanal, and the variant of the invention comprises the amino acid substitution D6N or D6H.

In another particular embodiment, the aldehyde is propanal, the ketone is propanone and the variant of the invention comprises the amino acid substitution D6E.

In another particular embodiment, the aldehyde is butanal, the ketone is propanone and the variant of the invention comprises the amino acid substitution D6E.

In another particular embodiment, the aldehyde is pentanal, the ketone is propanone and the variant of the invention comprises the amino acid substitution D6E.

In another particular embodiment, the aldehyde is isopentanal, the ketone is propanone and the variant of the invention comprises the amino acid substitution D6E.

In another particular embodiment, the aldol carboligation reaction takes place between two aldehydes, wherein both aldehydes are propanal and the variant of the invention comprises the am ino acid substitutions D6A and T26I.

In another particular embodiment, the aldol carboligation reaction takes place between two aldehydes, wherein both aldehydes are butanal and the variant of the invention comprises the amino acid substitutions D6A and T26L.

In one of the most preferred embodiments of the present invention, the aldehyde is N-Cbz-3-am inopropanal, the ketone is propanone and the variant of the invention comprises the amino acid substitution D6N.

In other of the most preferred embodiments of the present invention, the aldehyde is 3-hydroxypropanal, the ketone is propanone and the variant of the invention comprises the amino acid substitution D6H.

In another preferred embodiment of the method of the invention, this method further comprises an additional step c), at the end of the method, comprising a cyclation reaction of the product obtained in step (b). More preferably, this cyclation reaction comprises a reductive amination when the product obtained in step (b) comprises an amine group. Alternatively, this additional step of the method of the invention may be a step a′), taking place between steps (a) and (b).

Unless otherwise defined, all technical and scientific terms used herein have the same meaning as commonly understood by one of ordinary skilled in the art to which this invention belongs. Methods and materials similar or equivalent to those described herein can be used in the practice of the present invention. Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. The following examples and sequence listing are provided by way of illustration and are not intended to be limiting of the present invention.

### EXAMPLES

The following examples show illustrative reactions catalyzed by the FSA variants of the invention.

### Example 1

### General Procedure for Additions of Propanone (1a) to Hydroxyaldehydes

Reactions were conducted in a 50 mL screw capped conical-bottom polypropylene tubes. Method 1. To an enzyme (3 mg mL⁻¹) solution in plain water (12.8 mL), 1 M triethanolamine buffer, pH 8 (1 mL) was added. To this solution, aldehyde (80 mM, 3.2 mL of 500 mM aqueous stock solution) and propanone (3 mL) were added. Method 2. To an enzyme solution (3 mg m L⁻¹) in plain water (16 mL), 1 M triethanolamine buffer, pH 8 (1 mL) was added. To this solution, benzyloxyethanal (80mM) dissolved in propanone (3 mL) was added. For both Methods: Total reaction volume was 20 mL. Reaction mixtures were shaken (1000 rpm) at 25° C. for 24 h. After that, the reactions were stopped with MeOH (30 mL) to precipitate the enzyme. Method 1. The mixture was filtered through Celite, the MeOH was evaporated and the aqueous residue was lyophilized. Method 2. The mixture was filtered through activated charcoal. MeOH was evaporated and the reaction was lyophilized.

### Example 1.1

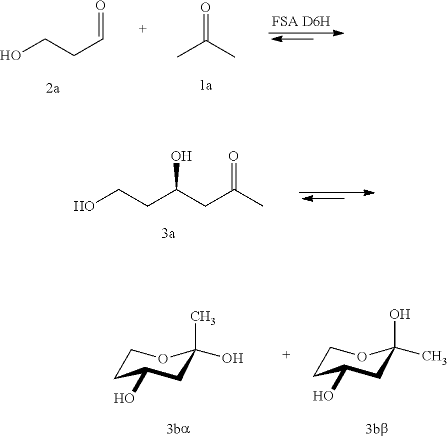

2-Methyltetrahydro-2H-pyran-2,4-diol (3bα=(2S,4R), 3bβ=(2R,4R) and (R)-4,6-dihydroxyhexan-2-one (3a): This compounds were obtained following the procedure described above (Method 1) as a mixture of anomers (3bα and 3bβ) and in equilibrium (slow) with the acyclic precursor (3). After enough time it can be found 3bβ as major product. 3-Hydroxypropanal (2a) (1.6 mmol) and FSA D6H were used. The product was purified by silica gel column chromatography and eluted with a step gradient of CHCl₃:MeOH from 1:0 to 93:7, yielding 50.0 mg (32%) of a mixture (3bα:3bβ:3. [α]_{D}²⁰=−24.2 (c=1.07 in MeOH); (3bα): ¹H NMR (500 MHz, CD₃OD) δ3.94 (m, 2H), 3.66 (ddd, J=11.6, 5.3, 1.8 Hz, 1H), 3.58 (ddd, J=12.8, 11.4, 2.3 Hz, 1H), 2.05 (ddd, J=12.6, 4.8, 2.1 Hz, 1H), 1.82 (ddt, J=10.4, 4.6, 2.2 Hz, 1H), 1.41 (m, 1H), 1.37 (s, 3H), 1.28 (m, 1H). ¹³C NMR (126 MHz, CDl₃OD) δ100.8, 65.0, 60.6, 45.9, 35.6, 23.9. (3bβ): ¹H NMR (400 MHz, CD₃OD) δ3.99 (m, 1H), 3.56 (m, 1H), 3.49 (m, 1H), 2.09 (m, 1H), 2.06 (m, 1H), 1.71 (m, 1H), 1.65 (m, 1H), 1.33 (s, 3H). ¹³C NMR (126 MHz, CD₃OD) δ96.3, 65.1, 57.8, 46.0, 38.3, 24.3. Open form 3: ¹H NMR (500 MHz, CD₃OD) δ4.25-4.12 (m, 1H), 4.00-3.85 (m, 2H), 2.61 (dd, J=6.4, 2.8 Hz, 2H), 2.18 (s, 3H), 1.74-1.62 (m, 2H). ¹³C NMR (101 MHz, CD₃OD) δ210.5, 66.4, 59.8, 51.9, 40.6, 30.6. ESI-TOF: calculated for [M+Na]⁺ C₆H₁₂O₃Na: 155.0684, found 155.0689.

### Example 1.2

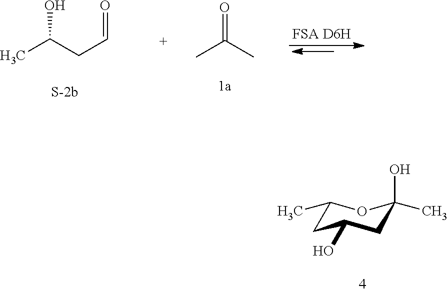

(2R,4R,6S)-2,6-dimethyltetrahydro-2H-pyran-2,4-diol (4): This corn pound was obtained following the procedure described above (Method 1). S-3-Hydroxybutanal (S-2b) (1.6 mmol) and FSA D6H were used. The product was purified by silica gel column chromatography and eluted with a step gradient of CHCl₃:MeOH from 1:0 to 95:5, yielding 60.7 mg (25%) of 4. [α]_{D}²⁰=−58.8 (c=1.12 in MeOH); ¹H NMR (400 MHz, CD₃OD) δ3.96 (tt, J=11.2, 4.7 Hz, 1H), 3.67 (ddd, J=11.5, 6.4, 2.1 Hz, 1H), 2.03 (ddd, J=12.5, 4.8, 1.9 Hz, 1H), 1.89 (ddt, J=12.3, 4.4, 2.1 Hz, 1H), 1.29 (s, 3H), 1.25 (dd, J=12.5, 11.2 Hz, 1H), 1.17 (d, J=6.3 Hz, 3H), 1.05 (dd, J=12.3, 11.4 Hz, 1H). ¹³C NMR (101 MHz, CD₃OD) δ66.5, 65.2, 45.3, 43.0, 24.1, 21.7. ESI-TOF: calculated for [M+Na]⁺ C₇H₁₄O₃Na: 169.0840, found 169.0837.

### Example 1.3

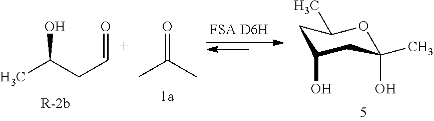

(2S,4R,6R)-2,6-dimethyltetrahydro-2H-pyran-2,4-diol (5): This compound was obtained following the procedure described above (Method 1). (R)-3-Hydroxybutanal (R-2b) (1.6 mmol) and FSA D6H were used. The product was purified by silica gel column chromatography and eluted with a step gradient of CHCl₃:MeOH from 1:0 to 95:5, yielding 21.2 mg (10%) of 5. [α]_{D}²⁰=+11.1 (c=0.36 in MeOH); ¹H NMR (500 MHz, CD₃OD) δ4.03 (m, 1H), 4.04 (m, 1H), 1.93 (ddd, J=14.4, 2.9, 2.1 Hz, 1H), 1.73 (ddt, J=13.7, 3.2, 2.2 Hz, 1H), 1.70-1.65 (m, 1H), 1.48-1.39 (m, 1H), 1.30 (s, 3H), 1.20 (d, J=6.3 Hz, 3H). ¹³C NMR (101 MHz, CD₃OD) δ100.0, 64.7, 60.8, 39.8, 38.8, 22.9, 20.1. ESI-TOF: calculated for [M+Na]⁺ C₇H₁₄O₃Na: 169.0840, found 169.0842.

### Example 1.4

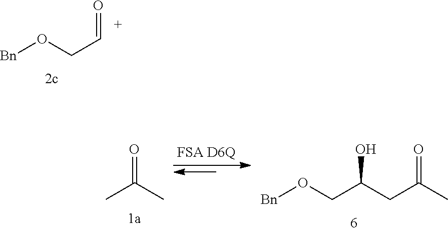

5-(benzyloxy)-4-hydroxypentan-2-one (6): This compound was obtained following the procedure described above (Method 2). Benzyloxyethanal (2c) (240 μL, 1.6 mmol) and FSA D6Q were used. After 24 h an additional amount of FSA D6Q (3 mg ml⁻¹) was added and the reaction was shaken (1000 rpm) at 25° C. for 24 h more. The product was purified by silica gel column chromatography and eluted with a step gradient of hexane:AcOEt from 1:0 to 1:1, yielding 256 mg (77%) of 6. [α]_{D}²⁰=−5.29 (c=1.26 in MeOH); HPLC: 10 to 100% of B in 30 min, t_{R}=16 min; ¹H NMR (400 MHz, CD₃OD) δ7.45-7.10 (m, 5H), 4.53 (d, J=1.9 Hz, 2H), 4.27-4.17 (m, 1H), 3.50-3.37 (m, 2H), 2.72-2.54 (m, 2H), 2.16 (s, 3H). ¹³C NMR (101 MHz, CD₃OD) δ210.1, 138.4, 127.9, 127.4, 127.2, 75.0, 74.3, 67.8, 49.6, 30.7.

### Example 2

### Scale Up of Aldol Addition of Cyclobutanone and Cyclopentanone to Hydroxyaldehydes

### General Procedure:

The reaction was conducted in a 50 mL screw capped conical-bottom polypropylene tubes. Method 1. To an enzyme (3 mg mL⁻¹) solution in plain water (14.8 mL), 1 M triethanolamine buffer, pH 8 (1 mL) was added. To this solution, the aldehyde (80 mM, 3.2 mL of 500 mM aqueous stock solution) and ketone (1 mL) were added. Method 2. To an enzyme (3 mg mL⁻¹) solution in plain water (18 mL), 1 M triethanolamine buffer, pH 8 (1 mL) was added. To this solution, aldehyde (80 mM) and ketone (1 mL) were added. Methods 1 and 2: Total reaction volume was 20 mL. Reaction mixtures were shaken (1000 rpm) at 25° C. for 24 h. After that, the reaction was stopped with MeOH (30 mL) to precipitate the enzyme. Method 1: the mixture was filtered through activated charcoal. Method 2 the mixture was filtered through Celite. Methods 1 and 2: MeOH was evaporated and the reaction was lyophilized.

### Example 2.1

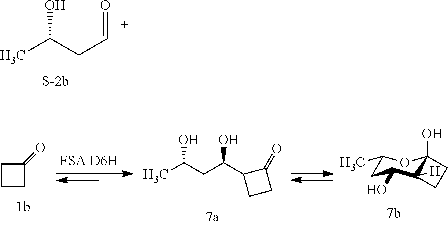

(1R,3S,5R,6R)-3-methyl-2-oxabicyclo[4.2.0]octane-1,5-diol (7b) in equilibrium with its acyclic form 7a: This compound was obtained following the procedure described above (Method 1). (S)-3-Hydroxybutanal (S-2b) (1.6 mmol), cyclobutanone (1b) and FSA D6H were used. The product was purified by silica gel column chromatography and eluted with a step gradient of CHCl₃:MeOH from 1:0 to 95:5, yielding 55.3 mg (22%) of 7b. [α]_{D}²⁰=+13.69 (c=2.0 in MeOH); ¹H NMR (400 MHz, CD₃OD) δ3.93 (dt, J=12.0, 6.3 Hz, 1H), 3.72 (dqd, J=11.1, 6.3, 1.4 Hz, 1H), 2.65 (ddd, J=9.9, 8.9, 6.5 Hz, 1H), 1.96-1.89 (m, 2H), 1.73 (dddd, J=12.7, 5.9, 1.5, 0.7 Hz, 1H), 1.64-1.53 (m, 2H), 1.36 (dd, J=12.6, 11.4 Hz, 1H), 1.20 (d, J=6.3 Hz, 3H). ¹³C NMR (101 MHz, CD₃OD) δ67.6, 65.7, 46.8, 38.3, 34.8, 21.8, 13.3. (7a) (R)-2-((1R,3S)-1,3-dihydroxybutyl)cyclobutan-1-one: ¹H NMR (400 MHz, CD₃OD) δ3.94 (m, 2H), 3.39 (dddd, J=9.7, 7.1, 5.3, 2.7 Hz, 1H), 3.02-2.80 (m, 2H), 2.18-2.04 (m, 1H), 2.02-1.95 (m, 1H), 1.71 (m, 1H), 1.55-1.48 (m, 1H) 1.18 (d, J=1.7 Hz, 3H). ¹³C NMR (101 MHz, CD₃OD) δ68.4, 67.5, 65.1, 45.6, 45.1, 24.3, 14.3. ESI-TO F: calculated for [M+Na]⁺ C₈H₁₄O₃Na: 181.0840, found 181.0843.

### Example 2.2

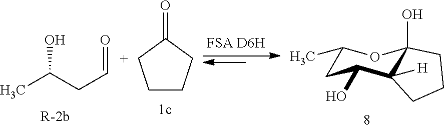

(2S,4R,4aR,7aR)-2-methylhexahydrocyclopenta[b]pyran-4,7a(2H)-diol (8): This compound was obtained following the procedure described above (Method 1). (S)-3-Hydroxybutanal (S-2b) (1.6 mmol), cyclopentanone (1c) and FSA D6H were used. The product was purified by silica gel column chromatography and eluted with a step gradient of CHCl₃:MeOH from 1:0 to 95:5, yielding 22.6 mg (8%) of 8. [α]_{D}²⁰=−2.03 (c=1.49 in MeOH); ¹H NMR (400 MHz, CD₃OD) δ4.05 (dt, J=11.8, 5.4 Hz, 1H), 3.71 (dqd, J=12.7, 6.3, 2.1 Hz, 1H), 2.20 (h, J=5.6 Hz, 1H), 1.94-1.86 (m, 1H), 1.78-1.57 (m, 6H), 1.43-1.30 (m, 1H), 1.18 (d, J=6.3 Hz, 3H). ¹³C NMR (126 MHz, CD₃OD) δ109.5 (d, J=9.0 Hz), 65.9, 65.6, 47.1, 35.6, 34.6, 21.6, 20.3, 19.4. ESI-TOF: calculated for [M+Na]⁺ C₉H₁₆O₃Na: 195.0997, found 195.0991.

### Example 2.3

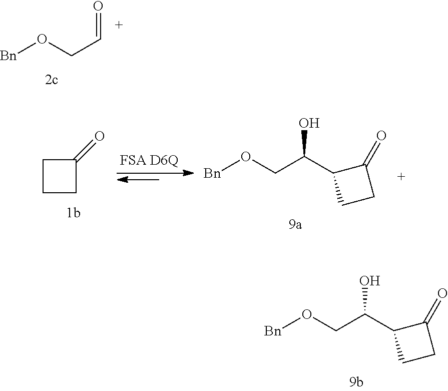 [SMILES: O=CCOCc1ccccc1 ; O=C1CCC1 ; O=C1CC[C@@H]1[C@H](O)COCc1ccccc1 ; O=C1CC[C@@H]1[C@@H](O)COCc1ccccc1]

(R)-2-((S)-2-(benzyloxy)-1-hydroxyethyl)cyclobutan -1-one (9a) (75%). This compound was obtained following the procedure described above (Method 2). Benzyloxyethanal (2c) (1.6 mmol), cyclobutanone (1b) and FSA D6Q were used. The product was purified by silica gel column chromatography and eluted with a step gradient of hexane:AcOEt from 1:0 to 3:7, yielding 46.0 mg (13%) of product. HPLC: 10 to 100% of B in 30 min, t_{R}=18 min; ¹H NMR (400 MHz, CD₃OD) δ7.38-7.26 (m, 5H), 4.53 (s, 2H), 3.92 (dt, J=6.5, 5.0 Hz, 1H), 3.59 (dd, J=9.8, 6.6 Hz, 1H), 3.55 (m, 1H), 3.53 (m, 1H), 2.93 (ddd, J=10.1, 7.7, 2.7 Hz, 1H), 2.88 (ddd, J=9.8, 6.2, 2.5 Hz, 1H), 2.15-1.96 (m, 2H). ¹³C NMR (101 MHz, CD₃OD) δ212.0, 139.6, 129.3, 128.9, 128.7, 74.3, 73.5, 70.3, 64.1, 45.8, 13.9. Minor (R)-2-((R)-2-(benzyloxy)-1-hydroxyethyl)cyclobutan-1-one (9b) (25%): ¹H NMR (400 MHz, CD₃OD) δ7.38-7.26 (m, 5H), 4.53 (s, 2H), 4.05 (dt, J=6.3, 5.0 Hz, 1H), 3.47 (dd, J=9.0, 5.2, 1H), 3.40 (dd, J=9.8, 6.3, 1H), 3.05 (m, 1H), 2.81 (m, 1H), 2.15-1.96 (m, 2H). ¹³C NMR (101 MHz, CD₃OD) δ212.3, 139.6, 129.3, 128.9, 128.7, 74.3, 73.6, 69.1, 64.3, 46.0, 13.1. ESI-TO F: calculated for [M+Na]⁺ C₁₃H₁₆O₃Na: 243.0997, found 243.0990.

### Example 3

### Scale Up of Aldol Addition of Ethanal (1d) to Hydroxyaldehydes

### General Procedure:

The reaction was conducted in a 50 mL screw capped conical-bottom polypropylene tubes. To an enzyme (3 mg mL⁻¹) solution in plain water (15.7 mL), 1 M triethanolamine buffer, pH 8 (1 mL) was added. To this solution, aldehyde (80 mM, 3.2 mL of 500 mM aqueous stock solution) and ethanal (5%, 1 mL, using electrophile 2a or 100 mM, 112 μL using electrophiles 2b) were added. Total reaction volume was 20 mL. Reaction mixtures were shaken (1000 rpm) at 25° C. for 24 h. After that, the reaction was stopped with MeOH (30 mL) to precipitate the enzyme, and the mixture was filtered through activated charcoal. MeOH was evaporated and the reaction was lyophilized.

### Example 3.1

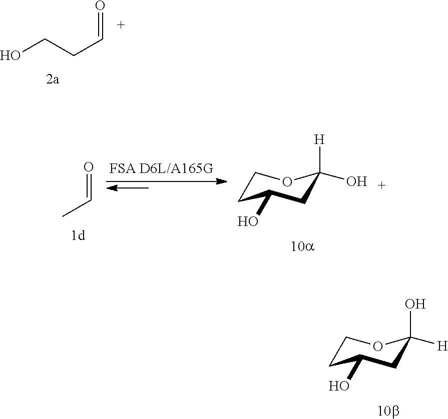

(2S,4R)-tetrahydro-2H-pyran-2,4-diol (10α-anomer) and (2R,4R)-tetrahydro-2H-pyran-2,4-diol (7a) (10β-anomer): These compounds were obtained as a mixture (≈1:1) following the procedure described above. 3-Hydroxypropanol (2a) (1.6 mmol) and FSA D6L/A165G were used. The product was purified by silica gel column chromatography and eluted with a step gradient of CHCl₃:MeOH from 1:0 to 95:5, yielding 41.5 mg (22%) of 10α:10β. [α]_{D}²⁰=−2.93 (c=0.62 in MeOH). (10α-Anomer): ¹H NMR (400 MHz, CD₃OD) δ5.15 (dd, J=4.4, 3.1 Hz, 1H), 4.06 (dd, J=8.4, 4.4 Hz, 1H), 3.87 (dd, J=8.8, 3.2 Hz, 1H), 3.72 (dd, J=5.6, 4.3 Hz, 1H), 1.85-1.76 (m, 2H), 1.58 (ddd, J=12.9, 8.5, 3.1 Hz, 1H), 1.49 (ddd, J=12.9, 8.6, 4.1 Hz, 1H). ¹³C NMR (101 MHz, CD₃OD) δ91.9, 63.1, 58.6, 39.6, 33.9. (10β-Anomer): ¹H NMR (400 MHz, CD₃OD) δ4.61 (dd, J=9.0, 2.2 Hz, 1H), 3.98-3.93 (m, 1H), 3.72 (m, 1H), 3.40 (td, J=11.9, 2.5 Hz, 1H), 2.07 (ddt, J=12.1, 4.2, 2.0 Hz, 1H), 1.77 (m, 1H), 1.39 (m, 1H), 1.29 (m, 1H).¹³C NMR (101 MHz, CD₃OD) δ94.2, 66.0, 61.1, 41.8, 34.1. ESI-TOF: calculated for [M+2Na]⁺ C₅H₁₀O₃Na₂: 165.0503, found 165.0507.

### Example 3.2

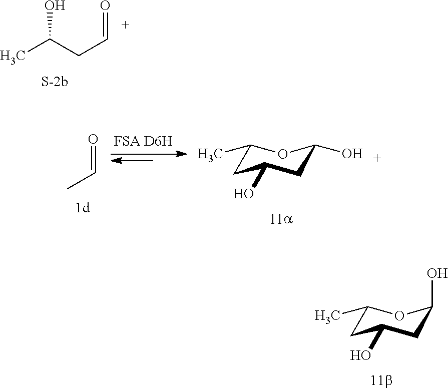

(2S,4R,6S)-6-methyltetrahydro-2H-pyran-2,4-diol (11α-anomer) (2R,4R,6S)-6-methyltetrahydro-2H-pyran-2,4-diol (11β-anomer). These compounds were obtained as a mixture (≈1:1) following the procedure described above. S-3-Hydroxybutanal (S-2b) (1.6 mmol) and FSA D6H were used. The product was purified by silica gel column chromatography and eluted with a step gradient of CHCl₃:MeOH from 1:0 to 95:5, yielding 57.1 mg (27%) of 11α:11β. [α]_{D}²⁰=−15.68 (c=0.19 in MeOH). (11α-anomer): ¹H NMR (400 MHz, CD₃OD) δ4.65 (dd, J=9.7, 2.1 Hz, 1H), 3.80-3.70 (m, 1H), 3.52 (ddd, J=11.2, 6.3, 2.0 Hz, 1H), 2.09 (ddt, J=12.0, 4.3, 2.0 Hz, 1H), 1.86 (ddt, J=12.4, 4.2, 1.9 Hz, 1H), 1.22 (d, J=6.2 Hz, 3H), 1.12-1.00 (m, 2H). ¹³C NMR (101 MHz, CD₃OD) δ92.0, 67.9, 66.0, 41.7, 41.6, 20.2. (11β-anomer): ¹H NMR (400 MHz, CD₃OD) δ5.29 (d, J=3.6 Hz, 1H), 4.17-3.98 (m, 2H), 2.02-1.90 (m, 3H), 1.40 (ddd, J=12.4, 11.4, 3.6 Hz, 1H), 1.16 (d, J=6.3 Hz, 3H). ¹³C NMR (101 MHz, CD₃OD) δ93.9, 63.5, 62.6, 42.5, 39.2, 20.4. ESI-TOF: calculated for [M+H]⁺ C₆H₁₃O₃: 133.0864, found 133.0860.

### Example 3.3

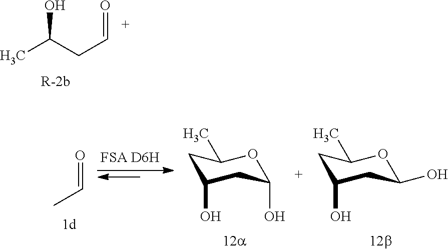

(2S,4R,6R)-6-methyltetrahydro-2H-pyran-2,4-diol (12α-anomer) (2R,4R,6R)-6-methyltetrahydro-2H-pyran-2,4-diol (12β-anomer). These compounds were obtained as a mixture of α:β anomers 4:1 following the procedure described above. R-3-Hydroxybutanal (R-2b) (1.6 mmol) and FSA D6H were used. The product was purified by silica gel column chromatography and eluted with a step gradient of CHCl₃:MeOH from 1:0 to 95:5, yielding 28.7 mg (14%) of product 12α:12β. [α]_{D}²⁰=−8.5 (c=1.7 in MeOH);(12α-anomer) ¹H NMR (400 MHz, CD₃OD) δ5.02 (dd, J=9.9, 2.2 Hz, 1H), 4.17 (t, J=3.0 Hz, 1H), 3.99 (dqd, J=12.6, 6.3, 2.2 Hz, 1H), 1.83 (ddt, J=13.5, 3.1, 2.1 Hz, 1H), 1.63-1.55 (m, 1H), 1.43 (ddd, J=13.3, 9.9, 3.0 Hz, 1H), 1.38-1.30 (m, 1H), 1.15 (d, J=6.3 Hz, 3H). ¹³C NMR (101 MHz, CD₃OD) δ91.7, 66.4, 64.8, 39.0, 39.0, 20.3. (12β-anomer) ¹H NMR (400 MHz, CD₃OD) δ5.14 (t, J=3.0 Hz, 1H), 4.36 (ddd, J=10.8, 6.5, 2.8 Hz, 1H), 4.11-4.08 (m, 1H), 1.77 (td, J=3.4, 2.9, 1.4 Hz, 1H), 1.76-1.69 (m, 1H), 1.53-1.48 (m, 1H), 1.15 (d, J=6.3 Hz, 3H). ¹³C NMR (101 MHz, CD₃OD) δ92.1, 64.2, 59.5, 39.3, 35.5, 20.1. ESI-TOF: calculated for [M+H]⁺ C₆H₁₃O₃: 133.0864, found 133.0865.

### Example 4

### Scale Up of Aldol Addition of Propanone, 2-Butanone, 3-Pentanone, Ciclobutanone and Cyclopentanone to N-Cbz-Aminoaldehydes

### General Procedure:

The reaction was conducted in a 50 mL screw capped conical-bottom polypropylene tubes (Falcon tubes). To an enzyme solution (3 mg mL⁻¹) in plain water (16 mL or 18 mL), 1 M triethanolamine buffer pH 8 (1 mL) was added. To this solution, the aldehyde (80 mM) dissolved in the ketones (3 mL acetone, 1 mL the rest) was added. Total reaction volume was 20 mL. Reaction mixtures were shaken (1000 rpm) at 25° C. for 24 h. After that, the reaction was stopped with MeOH (30 mL) to precipitate the enzyme. Then, the mixture was filtered through Celite, the MeOH was evaporated and the aqueous residue was lyophilized, purified characterized and submitted to reductive amination.

### Intramolecular Reductive Amination

General procedure: The aldol adduct (3 mM final concentration) was dissolved in H₂O:MeOH 1:1 (70 mL), then Pd/C (60 mg) was added. The mixture was shaken under H₂ (2.5 atm) overnight at room temperature. After that, the reaction was filtered and the solvent was evaporated. Products were characterized without any further purification.

### Example 4.1

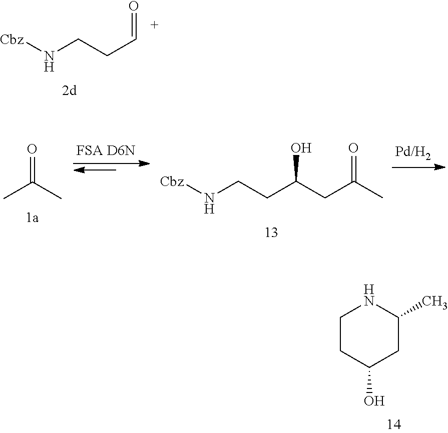

(R)-N-Cbz-6-amino-4-hydroxyhexan-2-one (13): This compound was obtained following the procedure described above. N-Cbz-3-aminopropanal (2d) (331.2 mg, 1.6 mmol) and FSA D6N were used. After 24 h an additional amount of FSA D6N (3 mg ml⁻¹) was added and the reaction was shaken (1000 rpm) at 25° C. for 24 h. The product was purified by silica gel column chromatography and eluted with a step gradient of hexane:AcOEt from 1:0 to 3:7, yielding 266.2 mg (85%) of the title compound. [α]_{D}²⁰=+10.74 (c=2.1 in MeOH); HPLC: 10 to 100% of B in 30 min, t_{R}=16 min; Chiral HPCL: t_{R}=25 min (racemic sample: t_{R}(R)=25 min, t_{R}(S)=30 min); ¹H NMR (400 MHz, CD₃OD) δ7.45-7.18 (m, 5H), 5.07 (s, 2H), 4.19-3.99 (m, 1H), 3.23 (t, J=7.0 Hz, 2H), 2.59 (d, J=6.4 Hz, 2H), 2.15 (s, 3H), 1.76-1.47 (m, 2H). ¹³C NMR (101 MHz, CD₃OD) δ210.4, 159.0, 138.4, 129.4, 128.9, 128.8, 67.4, 66.5, 51.7, 38.6, 38.2, 30.6.

The intramolecular reductive amination reaction produced: (2R,4R)-2-methylpiperidin-4-ol (14): ¹H NMR (400 MHz, CD₃OD) δ3.77 (tt, J=11.0, 4.5 Hz, 1H), 3.41-3.33 (m, 1H), 3.25-3.14 (m, 1H), 2.97 (td, J=13.4, 3.0 Hz, 1H), 2.09 (tdd, J=13.3, 4.7, 2.5 Hz, 2H), 1.55 (tdd, J=13.7, 11.0, 4.5 Hz, 1H), 1.39 (dd, J=13.1, 11.3 Hz, 1H), 1.32 (d, J=6.6 Hz, 3H). ¹³C NMR (101 MHz, CD₃OD) δ65.2, 51.4, 42.3, 39.5, 30.9, 18.0. ESI-TOF: calculated for [M+H]⁺ C₆H₁₄NO: 116.1075, found 116.1077.

### Example 4.2

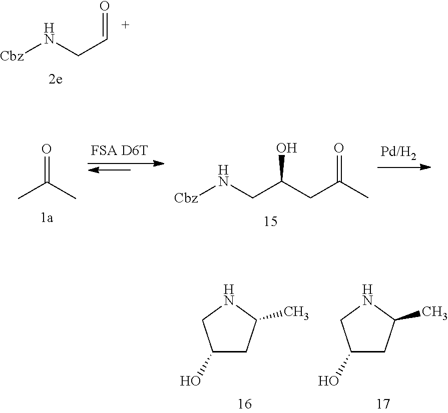

(S)-N-Cbz-5-amino-4-hydroxypentan-2-one (15): This compound was obtained following the procedure described above. N-Cbz-glycinal (2e) (310.4 mg, 1.6 mmol) and FSA D6T were used. The product was purified by silica gel column chromatography and eluted with a step gradient of Hexane:AcOEt from 1:0 to 3:7, yielding 114.8 mg (29%) of product. [α]_{D}²⁰=+3.4 (c=1.9 in MeOH); HPLC:10 to 100% of B in 30 min, t_{R}=15 min; Chiral HPCL: t_{R}=31 min (racemic sample: t_{R}(R)=31 min, t_{R}(S)=28 min); ¹H NMR (400 MHz, CD₃OD) δ7.34 (d, J=6.0 Hz, 5H), 5.08 (s, 2H), 4.18-4.04 (m, 1H), 3.16 (dd, J=5.8, 1.4 Hz, 2H), 2.64-2.52 (m, 2H), 2.15 (s, 3H). ¹³C NMR (101 MHz, CD₃OD) δ209.9, 159.1, 138.3, 129.4, 129.0, 128.8, 126.3, 68.0, 67.5, 48.4, 47.4, 30.6.

The intramolecular reductive amination reaction produced two diastereoisomers in a 70:30 ratio. Major: (3S,5R)-5-methylpyrrolidin-3-ol (16). ¹H NMR (400 MHz, CD₃OD) δ4.41-4.31 (m, 1H), 3.22 (p, J=7.0, 7.0, 6.9, 6.9 Hz, 1H), 2.92 (dd, J=12.2, 1.5 Hz, 1H), 2.87 (dd, J=12.2, 4.9 Hz, 1H), 2.27 (ddd, J=13.5, 7.8, 6.5 Hz, 1H), 1.32 (dddd, J=13.6, 7.8, 3.7, 1.2 Hz, 1H), 1.27 (d, J=6.5 Hz, 3H). ¹³C NMR (101 MHz, CD₃OD) δ71.8, 54.5, 54.0, 42.1, 19.3. Minor: (3S,5S)-5-methylpyrrolidin-3-ol (17). ¹H NMR (400 MHz, CD₃OD) δ4.41-4.29 (m, 1H), 3.45 (dp, J=10.0, 6.5, 6.5, 6.4, 6.4 Hz, 1H), 3.27 (dd, J=12.1, 5.2 Hz, 1H), 2.83-2.74 (m, 1H), 1.92 (ddt, J=13.4, 6.2, 1.4, 1.4 Hz, 1H), 1.48 (ddd, J=13.4, 10.0, 5.8 Hz, 1H), 1.20 (d, J=6.5 Hz, 3H). ¹³C NMR (101 MHz, CD₃OD) δ71.5, 54.0, 52.9, 42.8, 18.3. ESI-TOF: calculated for [M+H]⁺ C₅H₁₂NO: 102.0919, found 102.0210.

### Example 4.3

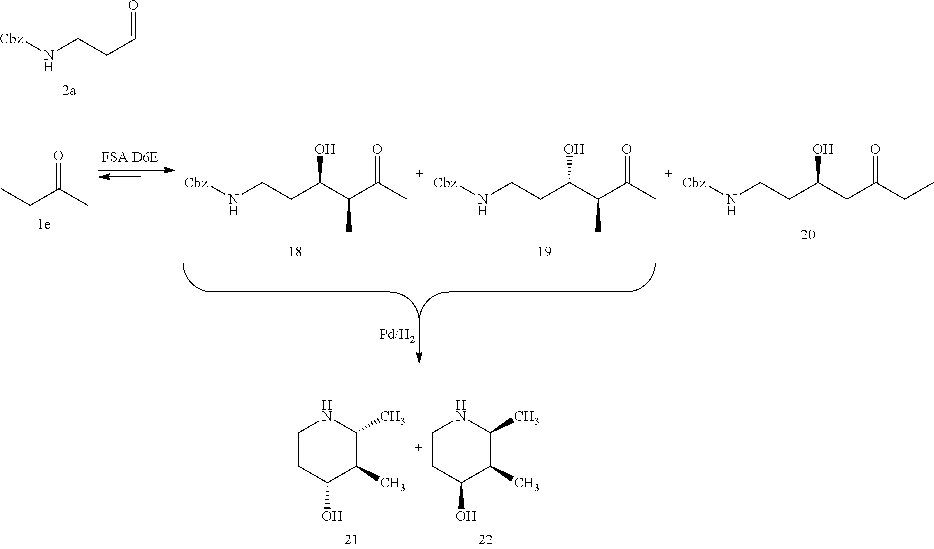

Major regioisomers: (3S,4R)-N-Cbz-6-Amino-4-hydroxy-3-methylhexan-2-one (18) and (3S,4S)-N-Cbz-6-Amino-4-hydroxy-3-methylhexan-2-one (19). This compound was obtained following the procedure described above. N-Cbz-3-aminopropanal (2a) (1.6 mmol), butanone (1e) and FSA D6E were used. The product was purified by silica gel column chromatography and eluted with a step gradient of hexane:AcOEt from 1:0 to 3:7, yielding 213 mg (48%) of 18+19, although 19 could not be assigned in the NMR spectra and the percentage formed during the aldol addition was inferred from the products obtained in the reductive amination. [α]_{D}²⁰=+33.5 (c=2.1 in MeOH); HPLC: 10 to 100% of B in 30 min, t_{R}=18 min; ¹H NMR (400 MHz, CD₃OD) δ7.50-7.19 (m, 5H), 5.05 (s, 2H), 3.90 (dt, J=9.2, 4.5 Hz, 1H), 3.26-3.15 (m, 2H), 2.67-2.53 (m, 1H), 2.14 (s, 3H), 1.57 (tdd, J=15.2, 7.7, 5.1 Hz, 2H), 1.06 (d, J=7.0 Hz, 3H). ¹³C NMR (101 MHz, CD₃OD) δ214.0, 158.2, 137.1, 128.7, 128.3, 128.1, 69.0, 65.9, 52.3, 37.3, 34.7, 28.2, 9.8. Minor regioisomer: (R)-N-Cbz-7-amino-5-hydroxyheptan-3-one (20). The product was purified by preparative HPLC and eluted with a step gradient of H₂O:CH₃CN from 1:0 to 1:1, flow rate 1 mL min⁻¹ and detection at 215 nm, yielding 25 mg of a mixture 44:56 of regioisomers I:II. [α]_{D}²⁰=+18.5 (c=0.9 in MeOH); HPLC: 10 to 100% of B in 30 min, t_{R}=19 min; ¹H NMR (400 MHz, CD₃OD) δ7.59-7.17 (m, 5H), 5.07 (s, 2H), 4.09 (dt, J=8.2, 4.0 Hz, 1H), 3.24 (q, J=7.0, 6.4 Hz, 2H), 2.59-2.54 (m, 2H), 2.49 (qd, J=7.3, 1.8 Hz, 2H), 1.76-1.60 (m, 2H), 1.01 (t, 3H). ¹³C NMR (101 MHz, CD₃OD) δ212.7, 159.0, 138.5, 129.2, 128.9, 128.8, 66.2, 65.3, 49.1, 36.8, 36.1, 34.6, 6.43.

The intramolecular reductive amination reaction of regioisomers 18+19 produced two diastereoisomers, 21 and 22, in a 87:13 ratio. Major: (2R,3R,4R)-2,3-dimethylpiperidin-4-ol (21): ¹H NMR (400 MHz, CD₃OD) δ3.36-3.22 (m, 2H), 2.96 (td, J=13.3, 3.1 Hz, 1H), 2.81 (dq, J=10.7, 6.5 Hz, 1H), 2.05 (ddt, J=13.7, 5.2, 2.8 Hz, 1H), 1.71-1.56 (m, 1H), 1.44-1.32 (m, 1H), 1.30 (d, J=6.5 Hz, 3H), 1.06 (d, J=6.5 Hz, 3H). ¹³C NMR (101 MHz, CD₃OD) δ70.6, 56.3, 43.6, 42.3, 31.6, 16.3, 12.4. Minor. (2S,3R,4S)-2,3-dimethylpiperidin-4-ol (22): ¹H NMR (500 MHz, CD₃OD) δ3.83 (dt, J=10.2, 4.4 Hz, 1H), 3.32-3.28 (m, 2H), 3.24 (d, J=12.0 Hz, 1H), 2.91-2.85 (m, 1H), 2.05-2.02 (m, 1H), 1.77-1.66 (m, 2H), 1.25 (d, J=6.8 Hz, 3H), 0.94 (d, J=7.2 Hz, 3H). ¹³C NMR (101 MHz, CD₃OD) δ68.3, 53.8, 42.7, 37.7, 26.6, 15.1, 4.8. ESI-TOF: calculated for [M+H]⁺ C₇H₁₆NO: 130.1231, found 130.1236.

### Example 4.4

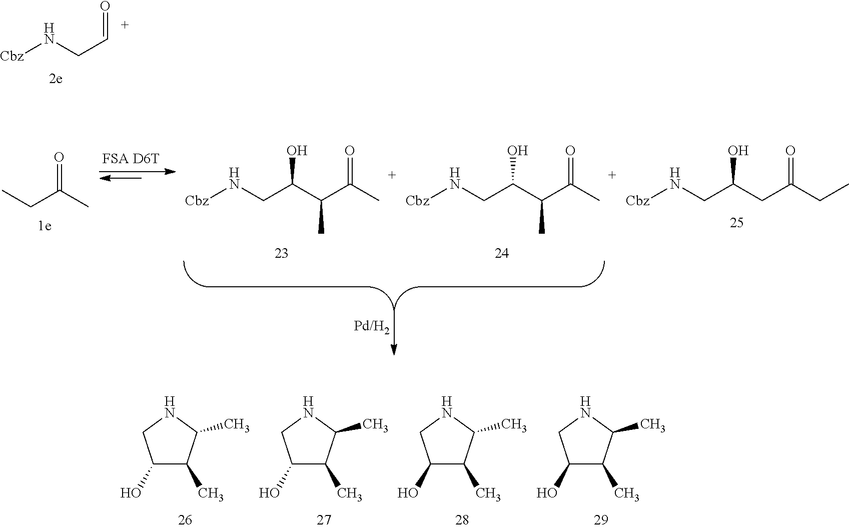

Major regioisomers: (3S,4S)-N-Cbz-5-amino-4-hydroxy-3-methylpentan-2-one (23) and (3S,4R)-N-Cbz-5-amino-4-hydroxy-3-methylpentan-2-one (24) (from the most substituted side of Nu). These compounds were obtained following the procedure described above. N-Cbz-glycinal (1e) (1.6 mmol), butanone and FSA D6T were used. The product was purified by silica gel column chromatography and eluted with a step gradient of hexane:AcOEt from 1:0 to 3:7, yielding 119.2 mg (28%) of 23+24. [α]_{D}²⁰=+13.0 (c=0.78 in MeOH); HPLC:10 to 100% of B in 30 min, t_{R}=17 min; ¹H NMR (400 MHz, CD₃OD) δ7.38-7.25 (m, 5H), 5.08 (s, 2H), 3.99 (q, J=5.8 Hz, 1H), 3.16 (qd, J=13.9, 6.1 Hz, 2H), 2.65 (dd, J=7.3, 5.6 Hz, 1H), 2.16 (s, 3H), 1.10 (d, J=7.0 Hz, 3H). ¹³C NMR (101 MHz, CD₃OD) δ213.6, (C═O carbamate and Cipso not detected), 129.4, 129.0, 128.9, 71.6, 67.5, 51.2, 45.8, 29.0, 11.0. Minor regioisomer (S)-N-Cbz-6-amino-5-hydroxyhexan-3-one (25). The product was purified by preparative HPLC and eluted with a step gradient of H₂O:CH₃CN from 100:0 to 50:50, flow rate 1 mL min⁻¹ and detection at 215 nm, yielding 12 mg of a 68:32 mixture of 23+25. [α]_{D}²⁰=+0.15 (c=0.8 in MeOH); HPLC:10 to 100% of B in 30 min, t_{R}=18 min; ¹H NMR (400 MHz, CD₃OD) δ7.45-7.09 (m, 5H), 5.05 (s, 2H), 4.09 (dt, J=11.6, 5.9 Hz, 1H), 3.17-3.10 (m, 2H), 2.54-2.50 (m, 2H), 2.46 (q, J=7.3 Hz, 2H), 0.98 (t, J=7.3 Hz, 3H). ¹³C NMR (101 MHz, CD₃OD) δ (C═O carbamate and carbonyl and Cipso not detected), 128.0, 127.6, 127.4, 66.6, 66.1, 46.3, 46.0, 36.0, 6.4.

The intramolecular reductive amination reaction of 23+24 produced four diastereoisomers in 80:8:8:4 rate. Major (80%). (3S,4R,5R)-4,5-dimethylpyrrolidin-3-ol (26): ¹H NMR (500 MHz, CD₃OD) δ3.99 (td, J=6.6, 4.9 Hz, 1H), 3.40 (dd, J=12.1, 6.9 Hz, 1H), 3.14 (dq, J=9.7, 6.6 Hz, 1H), 3.06-2.99 (m, 1H), 1.79-1.69 (m, 1H), 1.39 (d, J=6.6 Hz, 3H), 1.11 (d, J=6.9 Hz, 3H). ¹³C NMR (101 MHz, CD₃OD) δ75.2, 60.4, 50.1, 47.5, 15.3, 13.3. Minor (8%) (3S,4R,5S)-4,5-dimethylpyrrolidin-3-ol (27): ¹H NMR (500 MHz, CD₃OD) δ4.15 (dt, J=4.5, 2.0 Hz, 1H), 3.98-3.90 (m, 1H), 3.53-3.46 (m, 1H), 3.11-3.06 (m, 1H), 2.26-2.19 (m, 1H), 1.31 (d, J=7.0 Hz, 3H), 0.92 (d, J=7.5 Hz, 3H). ¹³C NMR (101 MHz, CD₃OD) δ75.6, 56.7, 50.5, 44.1, 11.6, 9.9. Minor (8%) (3R,4R,5R)-4,5-dimethylpyrrolidin-3-ol (28): ¹H NMR (500 MHz, CD₃OD) δ4.24 (t, J=4.1 Hz, 1H), 3.43-3.40 (m, 1H), 3.38-3.33 (m, 1H), 3.02-2.97 (m, 1H), 1.88-1.77 (m, 1H), 1.35 (d, J=7.1 Hz, 3H), 1.05 (d, J=2.6 Hz, 3H). ¹³C NMR (101 MHz, CD₃OD) δ71.8, 58.6, 52.1, 45.2, 13.7, 8.8. Minor (4%) (3R,4R,5S)-4,5-dimethylpyrrolidin-3-ol (29): ¹H NMR (500 MHz, CD₃OD) δ4.30 (td, J=4.7, 2.6 Hz, 1H), 3.84-3.75 (m, 1H), 3.35-3.27 (m, 1H), 3.19-3.14 (m, 1H), 2.35-2.30 (m, 1H), 1.33 (d, J=10.8 Hz, 3H), 1.05 (d, J=2.6 Hz, 3H). ¹³C NMR (101 MHz, CD₃OD) δ71.8, 57.2, 51.5, 40.0, 13.8, 7.1. ESI-TOF: calculated for [M+H]⁺ C₆H₁₄NO: 116.1075, found 116.1078.

### Example 4.5

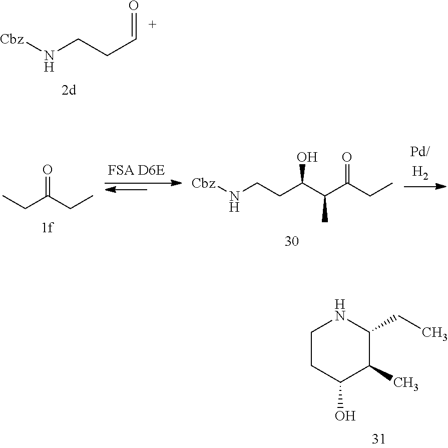

(4S,5R)-N-Cbz-7-amino-5-hydroxy-4-methylheptan-3-one (30): This compound was obtained following the procedure described above. N-Cbz-3-aminopropanal (2d) (1.6 mmol), 3-pentanone and FSA D6E were used. The product was purified by silica gel column chromatography and eluted with a step gradient of hexane:AcOEt from 1:0 to 3:7, yielding 164.1 mg (35%) of 30. [α]_{D}²⁰=+36.5 (c=2.3 in MeOH); HPLC: 10 to 100% of B in 30 min, t_{R}=20 min; ¹H NMR (400 MHz, CD₃OD) δ7.45-7.17 (m, 5H), 5.05 (s, 2H), 3.83 (ddd, J=9.4, 5.6, 3.7 Hz, 1H), 3.20 (hept, J=7.2 Hz, 2H), 2.62 (p, J=6.8 Hz, 1H), 2.52 (qd, J=7.2, 2.0 Hz, 2H), 1.67-1.39 (m, 2H), 1.08 (d, J=7.0 Hz, 3H), 0.99 (t, J=7.2 Hz, 3H). ¹³C NMR (101 MHz, CD₃OD) δ215.2, 157.6, 137.0, 128.0, 127.5, 127.3, 69.4, 65.9, 51.5, 37.5, 34.7, 34.5, 10.4, 6.5.

The intramolecular reductive amination reaction produced (2R,3R,4R)-2-ethyl-3-methylpiperidin-4-ol (31). ¹H NMR (400 MHz, CD₃OD) δ3.27-3.25 (m, 2H), 2.87 (td, J=13.1, 2.9 Hz, 1H), 2.56 (ddd, J=10.6, 7.4, 3.3 Hz, 1H), 2.02 (ddt, J=13.3, 5.1, 2.8 Hz, 1H), 1.85 (dqd, J=15.2, 7.6, 2.8 Hz, 1H), 1.66-1.49 (m, 2H), 1.38 (tq, J=10.2, 6.5 Hz, 1H), 1.05 (d, J=6.5 Hz, 3H), 1.00 (t, J=7.6 Hz, 3H). ¹³C NMR (101 MHz, CD₃OD) δ73.1, 62.9, 44.5, 43.3, 34.0, 25.3, 13.8, 9.3. ESI-TOF: calculated for [M+H]⁺ C₈H₁₈NO: 144.1388, found 144.1385

### Example 4.6

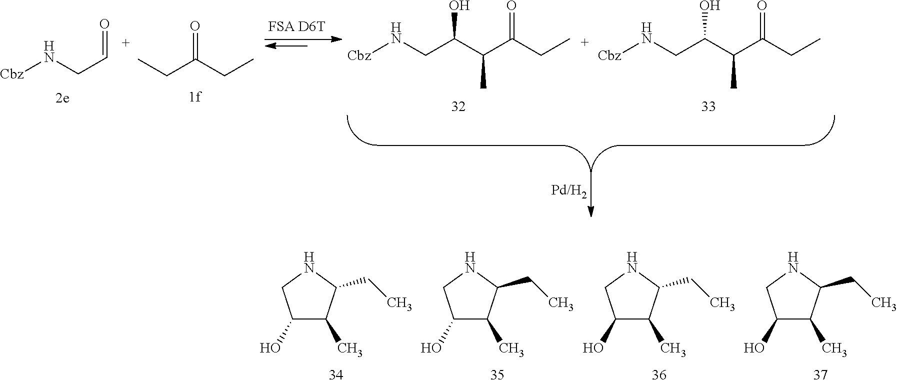

(4S,5S)-N-Cbz-6-amino-5-hydroxy-4-methyl-hexan-3-one (32) and (4S,5R)-N-Cbz-6-amino-5-hydroxy-4-methyl-hexan-3-one (33). These compounds were obtained following the procedure described above. N-Cbz-glycinal (2e) (1.6 mmol), 3-pentanone and FSA D6T were used. The product was purified by silica gel column chromatography and eluted with a step gradient of hexane:AcOEt from 1:0 to 3:7, yielding 134.4 mg (30%) of 32+33. [α]_{D}²⁰ =+12.3 (c=1.26 in MeOH); HPLC:10 to 100% of B in 30 min, t_{R}=19 min; ¹H NMR (400 MHz, CD₃OD) δ7.44-7.16 (m, 5H), 5.19-5.02 (m, 2H), 3.92 (q, J=5.6 Hz, 1H), 3.16-3.06 (m, 2H), 2.64 (dt, J=12.9, 6.8 Hz, 1H), 2.51 (q, J=7.2 Hz, 2H), 1.08 (d, J =7.0 Hz, 3H), 0.97 (t, J=7.2 Hz, 3H). ¹³C NMR (101 MHz, CD₃OD) δ214.6, 157.6, 136.9, 128.0, 127.5, 127.4, 70.5, 66.1, 49.0, 44.4, 34.3, 10.3, 6.5. Compound 33 was not clearly assigned in the NMR, but it was inferred from the structures observed in the reductive amination products.

The intramolecular reductive amination reaction produced four diastereoisomers in ≈79:8:5:8 rate. Major (79): (3S,4R,5R)-5-ethyl-4-methylpyrrolidin-3-ol (34). ¹H NMR (400 MHz, CD₃OD) δ3.97 (q, J=5.3 Hz, 1H), 3.36-3.30 (m, 1H), 3.02 (dd, J=12.0, 4.5 Hz, 1H), 2.91 (tdd, J=8.7, 5.1, 1.5 Hz, 1H), 1.91-1.75 (m, 2H), 1.72-1.61 (m, 1H), 1.10 (d, J=7.0 Hz, 3H), 1.03 (t, J=7.5 Hz, 3H). ¹³C NMR (101 MHz, CD₃OD) δ75.7, 66.7, 50.3, 46.3, 25.1, 14.5, 9.9. Minor (8): (3S,4R,5S)-5-ethyl-4-methylpyrrolidin-3-ol (35). ¹H NMR (400 MHz, CD₃OD) ¹H NMR (400 MHz, CD₃OD) δ4.24 (t, J=4.1 Hz, 1H), 3.41-3.34 (m, 1H), 3.17-3.12 (m, 2H), 1.89-1.82 (m, 2H), 1.54-1.43 (m, 1H), 1.10-1.09 (m, 3H), 1.02-0.98 (m, 3H). ¹³C NMR (101 MHz, CD₃OD) δ72.0, 64.8, 52.0, 43.6, 23.6, 15.5, 10.1. Minor (5): (3R,4R,5S)-5-ethyl-4-methylpyrrolidin-3-ol (36): ¹H NMR (400 MHz, CD₃OD) δ4.14 (d, J=4.5 Hz, 1H), 3.73-3.66 (m, 3H), 3.49-3.44 (m, 2H), 3.11-3.07 (m, 1H), 2.32-2.24 (m, 1H), 1.71-1.65 (m, 2H), 1.04 (d, J=7.5 Hz, 3H), 0.88 (d, J=7.5 Hz, 3H). ¹³C NMR (101 MHz, CD₃OD) δ75.7, 62.7, 50.6, 43.0, 22.1, 9.9, 9.5. Minor (8): (3R,4R,5R)-5-ethyl-4-methylpyrrolidin-3-ol (37): ¹H NMR (400 MHz, CD₃OD) δ4.35 (q, J=5.7 Hz, 1H), 3.47-3.43 (m, 1H), 3.27-3.23 (m, 1H), 3.08-3.04 (m, 1H), 2.38 (td, J=7.4, 5.0 Hz, 1H), 1.74-1.66 (m, 1H), 1.06 (d, J=5.1 Hz, 19H), 0.97 (d, J=5.2 Hz, 2H). ¹³C NMR (101 MHz, CD₃OD) δ71.3, 63.1, 49.7, 39.2, 25.9, 9.9, 6.7. ESI-TOF: calculated for [M+H]⁺ C₇H₁₆NO: 130.1231, found 130.1234

### Example 4.7

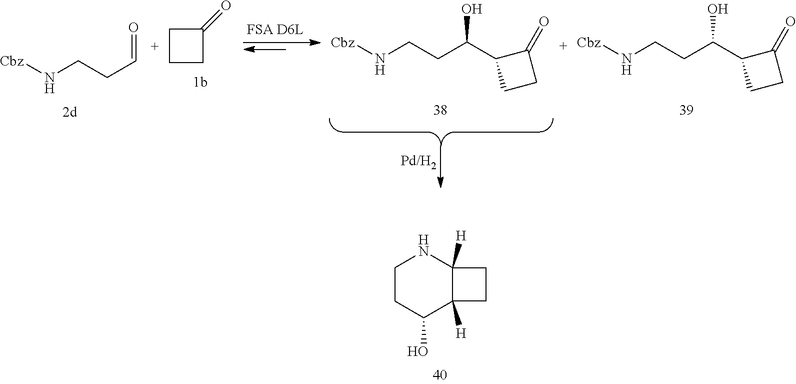

(R)-2-((R)-N-Cbz-3-amino-1-hydroxypropyl)cyclobutan-1-one (38) and (R)-2-((S)-N-Cbz-3-amino-1-hydroxypropyl)cyclobutan-1-one (39): These compounds were obtained following the procedure described above in a 80:20 ratio. N-Cbz-3-aminopropanal (2d) (1.6 mmol), cyclobutanone and FSA D6L were used. The product was purified by silica gel column chromatography and eluted with a step gradient of hexane:AcOEt from 1:0 to 3:7, yielding 201.8 mg (46%) of product. [α]_{D}²⁰=+36.49 (c=1.82 in MeOH); HPLC:10 to 100% of B in 30 min, t_{R}=17 min. Major (80%) (38): ¹H NMR (400 MHz, CD₃OD) δ7.41-7.25 (m, 5H), 5.06 (s, 2H), 3.73 (dt, J=9.0, 4.4 Hz, 1H), 3.47-3.35 (m, 1H), 3.23 (t, J=6.9 Hz, 2H), 3.03-2.78 (m, 2H), 2.09 (qd, J=10.3, 5.9 Hz, 1H), 1.96 (td, J=10.6, 5.2 Hz, 1H), 1.86-1.63 (m, 2H). ¹³C NMR (101 MHz, CD₃OD) δ212.3, 159.0, 138.4, 129.4, 129.0, 128.8, 69.1, 67.4, 66.9, 45.7, 38.7, 36.1, 14.4. Minor (20) (39): ¹H NMR (400 MHz, CD₃OD) δ7.41-7.25 (m, 5H), 5.06 (s, 2H), 3.94-3.87 (m, 1H), 3.21-3.10 (m, 1H), 3.22-3.15 (m, 2H), 3.01-2.80 (m, 2H), 2.14-1.90 (m, 2H), 1.71-1.52 (m, 2H). ¹³C NMR (101 MHz, CD₃OD) δ212.3, 159.0, 138.4, 129.4, 129.0, 128.8, 67.2, 67.1, 66.8, 45.9, 38.5, 36.5, 13.5. The intramolecular reductive am ination reaction produced (1R,2R,6R)-1,2,6-trimethylbicyclo[4.2.0]octane (40). ¹H NMR (400 MHz, CD₃OD) δ3.77 (dt, J=10.7, 5.7 Hz, 1H), 3.63-3.55 (m, 1H), 3.17 (dt, J=13.2, 4.2 Hz, 1H), 2.74 (t, J=7.1 Hz, 1H), 2.62 (ddd, J=13.7, 12.0, 2.9 Hz, 1H), 2.34-2.19 (m, 2H), 1.96-1.78 (m, 2H), 1.78-1.63 (m, 2H). ¹³C NMR (101 MHz, CD₃OD) δ64.2, 52.9, 40.3, 37.3, 28.1, 24.4, 18.4. ESI-TOF: calculated for [M+H]⁺ C₇H₁₄NO: 128.1075, found 128.1070

### Example 4.8

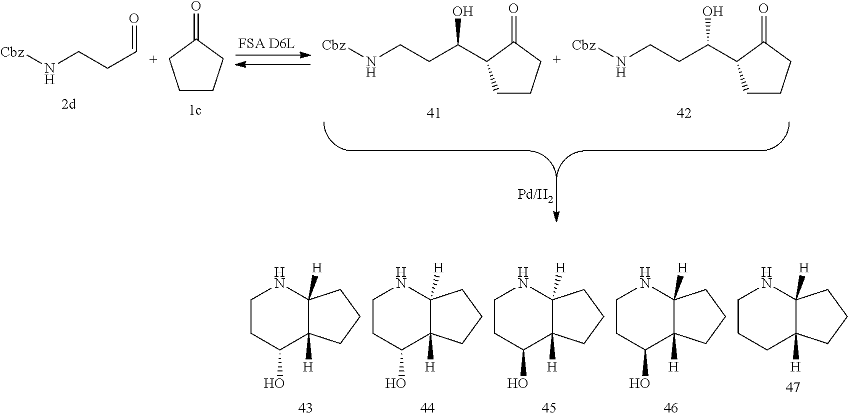

(R)-2-(((R)-N-Cbz-3-amino-1-hydroxypropyl)cyclopentan-1-one (41) and (R)-2-(((S)-N-Cbz-3-amino-1-hydroxypropyl)cyclopentan-1-one (42). These compounds were obtained following the procedure described above in a 67:33 ratio. N-Cbz-3-aminopropanal (2d) (1.6 mmol), cyclopentanone (1c) and FSA D6L were used. The product was purified by silica gel column chromatography and eluted with a step gradient of hexane:AcOEt from 1:0 to 3:7, yielding 303.5 mg (65%) of product. HPLC:10 to 100% of B in 30 min, t_{R}=18 and 19 min. Major (67%) (41): ¹H NMR (400 MHz, CD₃OD) δ7.41-7.17 (m, 5H), 5.06 (s, 2H), 3.78 (ddd, J=9.1, 5.4, 3.4 Hz, 1H), 3.27-3.12 (m, 2H), 2.35-2.21 (m, 2H), 2.18-2.09 (m, 3H), 1.83-1.71 (m, 3H), 1.70-1.61 (m, 1H). ¹³C NMR (101 MHz, CD₃OD) δ221.4, 157.5, 137.0, 128.0, 127.5, 127.3, 69.1, 66.1, 53.7, 38.7, 37.4, 25.8, 22.6, 20.2. Minor (33%) (42): ¹H NMR (400 MHz, CD₃OD) δ7.41-7.17 (m, 5H), 5.06 (s, 2H), 4.06 (ddd, J=8.4, 4.7, 3.2 Hz, 1H), 3.28-3.16 (m, 2H), 2.28-2.18 (m, 1H), 2.18-2.09 (m, 5H), 1.83-1.71 (m, 1H), 1.70-1.61 (m, 2H). ¹³C NMR (101 MHz, CD₃D) δ220.9, 157.5, 137.0, 128.0, 127.5, 127.3, 66.1, 66.8, 55.2, 38.9, 38.5, 36.3, 24.0, 21.5.

The intramolecular reductive amination reaction produced four diastereoisomers including a dehydroxylated analogue 47 in a 15:11:15:22:37 ratio. Selected signals of the NMR spectra. Minor (15%): (4R,4aS,7aR)-octahydro-1H-cyclopenta[b]pyridin-4-ol (43). ¹H NMR (500 MHz, MeOD) δ4.19 (dt, J=10.6, 5.2 Hz, 1H), 3.64 (m, 1H), 3.36 (m, 1H), 3.02 (m, 1H), 2.49 (m, 1H), 2.19-1.59 (m, 6H). ¹³C NMR (101 MHz, CD₃OD) 65.5, 59.0, 44.6, 42.3. Minor (11%): (4R,4aS,7aS)-octahydro-1H-cyclopenta[b]pyridin-4-ol (44). ¹H NMR (500 MHz, MeOD) δ3.99 (td, J=4.7, 3.1 Hz, 1H), 3.77 (m, 1H), 3.32 (m, 1H), 3.17 (dt, J=12.8, 4.4 Hz, 1H), 2.22 (m, 1H), 2.19-1.59 (m, 7H). ¹³C NMR (101 MHz, CD₃OD) δ63.2, 55.4, 45.4, 37.9. Minor (15%): (4S,4aS,7aS)-octahydro-1H-cyclopenta[b]pyridin-4-ol (45). ¹H NMR (500 MHz, MeOD) 0 3.62 (m, 1H), 3.50 (m, 1H), 3.08 (dd, J=13.6, 3.5 Hz, 1H), 2.90 (td, J=11.3, 7.1 Hz, 1H), 2.17 (m, 1H), 2.19-1.59 (m, 8H). ¹³C NMR (101 MHz, CD₃OD) δ70.5, 59.9, 49.7, 45.0. Minor (22%): (4S,4aS,7aR)-octahydro-1H-cyclopenta[b]pyridin-4-ol (46). ¹H NMR (500 MHz, MeOD) 0 3.48 (m, 1H), 3.47 (m, 1H), 3.01 (m, 1H), 2.78 (td, J=11.2, 7.1 Hz, 2H), 2.14 (m, 1H), 2.19-1.59 (m, 8H). ¹³C NMR (101 MHz, CD₃OD) δ61.4, 57.9, 43.9, 42.1. Major (37%) dehydroxylated analogue: (4aR,7aR)-octahydro-1H-cyclopenta[b]pyridine (47): ¹H NMR (500 MHz, MeOD) 0 3.58 (ddd, J=7.9, 5.4, 2.9 Hz, 4H), 3.27 (t, J=3.7 Hz, 2H), 3.01 (m, 1H), 2.35-2.25 (m, 1H), 2.11 (m, 1H), 1.79 (m, 2H), 1.85 (m, 2H), 1.71 (m, 1H), 1.76 (m, 2H). ¹³C NMR (101 MHz, CD₃OD) δ57.8, 42.3, 37.1, 28.2, 26.2, 22.4, 20.5, 17.8. ESI-TOF: calculated for [M+H]⁺ C₈H₁₆NO: 142.1231, found 142.1238

### Example 4.9

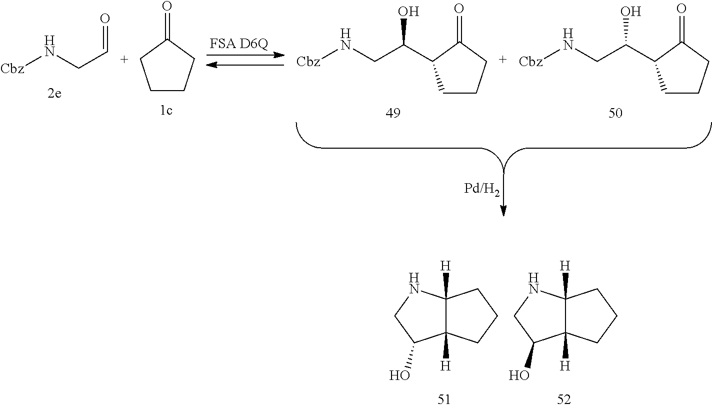

(R)-2-((S)-N-Cbz-2-amino-1-hydroxyethyl)cyclopentan-1-one (49) and (R)-2-((R)-N-Cbz-2-amino-1-hydroxyethyl)cyclopentan-1-one (50). These compounds were obtained following the procedure described above in a 50:50 ratio; HPLC: 10 to 100% of B in 30 min, t_{R}=17 and 18 min, respectively. N-Cbz-glycinal (x) (1.6 mmol), cyclopentanone and FSA D6Q were used. The product was purified by silica gel column chromatography and eluted with a step gradient of hexane:AcOEt from 1:0 to 3:7, yielding two fraction pools: fraction pool 1 containing 49 pure (53 mg,) and fraction pool 2 as a 1:2 mixture of 49:50 (117.2 mg), total 170.2 mg (38%) of product. (R)-2-((S)-N-Cbz-2-amino-1-hydroxyethyl)cyclopentan-1-one (49). ¹H NMR (400 MHz, CD₃OD) δ7.38-7.24 (m, 5H), 5.07 (s, 2H), 4.09 (td, J=6.9, 3.0 Hz, 1H), 3.23 (dd, J=13.7, 6.2 Hz, 1H), 3.14 (dd, J=13.7, 7.2 Hz, 1H), 2.33-2.15 (m, 2H), 2.15-1.92 (m, 4H), 1.83-1.70 (m, 1H). ¹³C NMR (101 MHz, CD₃OD) δ222.2, 159.0, 138.4, 129.4, 129.0, 128.9, 69.6, 67.5, 53.2, 46.3, 39.8, 23.7, 21.7. Fraction pool 2 49:50 33:67 ratio. NMR: (R)-2-((R)-N-Cbz-2-amino-1-hydroxyethyl)cyclopentan-1-one (50): ¹H NMR (400 MHz, CD₃OD) δ7.42-7.16 (m, 5H), 5.07 (s, 2H), 3.83 (q, J=5.5 Hz, 1H), 3.38-3.29 (m, 2H), 2.32-1.98 (m, 5H), 1.91-1.71 (m, 2H). ¹³C NMR (101 MHz, CD₃OD) δ222.5, 138.5, 129.4, 128.8, 70.8, 67.5, 52.6, 45.7, 40.1, 27.5, 21.8.

The intramolecular reductive amination reaction of the fraction pool 2 (49:50 33:67 ratio) produced two diastereoisomers the corresponding to the aldol adducts 51 and 52, respectively. From aldol adduct 49: (3S,3aS,6aR)-octahydrocyclopenta[b]pyrrol-3-ol (51). ¹H NMR (500 MHz, CD₃OD) δ4.35 (dt, J=6.7, 3.5 Hz, 1H), 3.99 (ddd, J=9.0, 7.2, 3.3 Hz, 1H), 3.15 (d, J=3.5 Hz, 2H), 2.81 (tdd, J=9.3, 6.4, 3.6 Hz, 1H), 2.00-1.92 (m, 1H), 1.93-1.86 (m, 2H), 1.89-1.75 (m, 2H), 1.67-1.62 (m, 1H). ¹³C NMR (101 MHz, CD₃OD) δ70.6, 64.6, 53.9, 47.1, 30.8, 26.0, 24.9. From aldol adduct 50: (3R,3aS,6aR)-octahydrocyclopenta[b]pyrrol-3-ol (52). ¹H NMR (500 MHz, CD₃OD) δ4.19-4.12 (m, 2H), 3.20-3.15 (m, 1H), 3.09 (dt, J=12.1, 1.6 Hz, 1H), 2.65 (ddd, J=9.6, 7.9, 6.3 Hz, 1H), 1.99-1.92 (m, 1H), 1.93-1.87 (m, 1H), 1.69-1.67 (m, 1H), 1.68-1.61 (m, 1H), 1.57-1.51 (m, 1H), 1.40 (dq, J=13.3, 6.7 Hz, 1H). ¹³C NMR (101 MHz, CD₃OD) δ75.6, 63.0, 51.7, 51.6, 31.7, 29.1, 24.9. ESI-TOF: calculated for [M+H]⁺ C₇H₁₄NO: 128.1075, found 128.1079.

### Example 5

### Scale Up of Aldol Addition of Ethanal

### Example 5.1

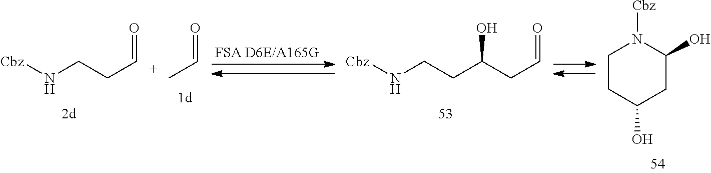

(2S,4R)-N-Cbz-piperidine-2,4-diol (54): The reaction was conducted in a 50 mL screw capped conical-bottom polypropylene tubes. To a FSA D6E/A165G (3 mg mL⁻¹) solution in plain water (18.9 mL), 1 M triethanolamine buffer, pH 8 (1 mL) was added. To this solution, N-Cbz-3-aminopropanal (331.2 mg, 80 mM) and acetaldehyde (100 mM, 112 μL) were added. Total reaction volume was 20 mL. Reaction mixtures were shaken (1000 rpm) at 25° C. for 24 h. After that, the reaction was stopped with MeOH (30 mL) to precipitate the enzyme, and the mixture was filtered through Celita MeOH was evaporated and the reaction was lyophilized. The product was purified by silica gel column chromatography and eluted with a step gradient of hexane:AcOEt from 1:0 to 3:7, yielding 191.3 mg (47%) of the title compound. [α]_{D}²⁰=+8.51 (c=2.1 in MeOH); HPLC: 10 to 100% of B in 30 min, t_{R}=14.5 min; ¹H NMR (400 MHz, CD₃OD) δ7.59-7.24 (m, 5H), 5.58-5.47 (m, 1H), 5.22-4.99 (m, 2H), 4.11-4.06 (m, 1H), 4.03 (tt, J=11.3, 4.5 Hz, 1H), 3.12-2.98 (m, 1H), 2.24-2.13 (m, 1H), 2.02-1.92 (m, 1H), 1.94-1.84 (m, 1H), 1.49-1.42 (m, 1H), 1.42-1.33 (m, 1H). ¹³C NMR (101 MHz, CD₃OD) δ155.7, 136.5, 128.2, 127.8, 127.5, 83.0, 67.1, 63.4, 38.7, 37.0, 35.1, 31.2. Rotamer: ¹H NMR (400 MHz, CD₃OD) δ7.59-7.24 (m, 5H), 5.49-5.43 (m, 1H), 5.16-5.08 (m, 2H), 4.03-3.99 (m, 1H), 3.97-3.91 (m, 1H), 3.45-3.38 (m, 1H), 2.12-2.04 (m, 1H), 1.86 (dt, J=14.7, 3.9 Hz, 1H), 1.81 - 1.70 (m, 2H). ¹³C NMR (101 MHz, CD₃OD) δ155.4, 136.5, 128.2, 127.8, 127.5, 82.5, 67.1, 60.0, 35.2, 33.0, 31.3. ESI-TOF: calculated for [M+H]⁺ C₁₃H₁₈NO₄: 252.1235, found 252.1231

### Example 6

### Aldol Addition Propanal to Formaldehyde

### Example 6.1

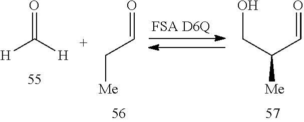

Reaction was conducted in 250 mL Erlenmeyer. To a FSA D6Q (3 mg mL⁻¹, 300 mg) solution in plain water (93 mL), 1 M triethanolamine buffer pH 8 (5 mL) was added. To this solution, formaldehyde (10 mmol, 813 μL of a 12.3 M solution) and propanal (10 mmol, 725 μL) were added. Total reaction volume was 99.5 m L. Reaction mixtures were shaken (200 rpm) at 25° C. for 48 h.

### Coupling Reaction to Structurally Characterize the Aldol Adduct 57.

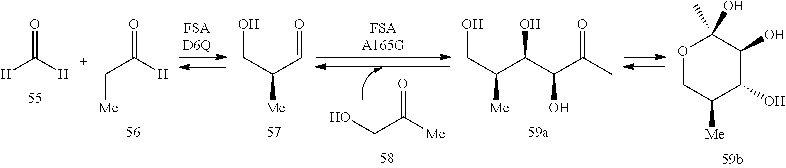

After 48 hours hydroxyacetone (58) (10 mmol, 680 μL) and FSA A165G (3 mg m L⁻¹, 300 mg) were added to the reaction and shaken (200 rpm) at 25° C. during an additional 24 hours. After that, the reaction was stopped with MeOH (100 mL) to precipitate the enzyme, and the mixture was filtered through activated charcoal. MeOH was evaporated and the reaction was lyophilized. The product was purified by silica gel column chromatography eluted with step gradient CHCl₃:MeOH from 1:0 to 95:5, yielding 392.2 mg (25%) of product (59b). Product 59b was structurally characterized by NMR.

Enzymatic Aldehyde Oxidation to Acid. Formation of 2-hydroxyisobutyrate (12).

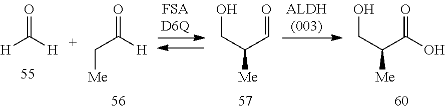

Aldehyde dehydrogenase from Prozomix (ALDH (003), KIT: Aldehyde dehydrogenase Panel, Cat. No.: PRO-ALDH (003) LOT: 2016-1) was used. To the reaction mixture containing 57 (12 mL, ≈100 mM of 11), NAD⁺ (226.36 μL from a 66.3 mM stock solution, final concentration 1 mM), and NOX (PROZO) (1254.48 μL from a 119.6 mg mL⁻¹ stock solution) were added (final concentration of 57=80 mM, total volume 15 mL). The reaction was started by adding AIDH (771.7 μL from a 194.4 mg mL⁻¹ stock solution) and the reaction placed in a shaker at 1000 rpm at room temperature. After 3 hours, the conversion of aldol adduct to 60 was approximately 96.4%.

HPLC analysis of propanal, formaldehyde and aldol adduct: Samples (5 μL) were withdrawn immediately after adding the enzyme and after 24 hours, and O-benzylhydroxylamine (50 μL, of a 0.13 mM stock solution in pyridine:methanol:water 33:15:2) and placed at room temperature for 5 min. After that, methanol (450 μL) was added and subsequently injected (30 μL) to the HPLC system. HPLC analysis: column: XBridge® C18, 5 μm, 4.6×250 mm column (Waters), solvent system A: 0.1% (v/v) TFA in H₂O; solvent B: 0.095% (v/v) TFA in CH₃CN:H₂O 80:20, gradient elution from 10 to 100% of B over 30 min, 30° C., flow rate 1 mL min⁻¹ and detection 215 nm.

2-Hydroxyisobutyrate (60) identification by reaction with 2,4′-dibromo-acetophenone: After the reaction was finished, MeOH was added (150 mL; 10×reaction volume) and the enzymes precipitated were filtered off using Celite. Methanol was then evaporated and the residue was lyophilized. The solid obtained was dissolved in DMF (10 mL) and (1.5 mmol) was added. After 7 hours of reaction, the mixture was diluted in ethyl acetate (50 m L), silica gel (50 g) was then added and solvent was evaporated to dryness. Product purification was carried by column chromatography (47×4.5 cm) on silica gel (100 g) with a gradient elution: hexane/ethyl acetate 100:0 (200 mL), 90:10 (200 mL); 80:20 (200 mL), 70:30 (200 mL) 60:40 (200 mL) and 50:50 (1 L), product eluted at 50:50. Fractions (10 mL) were analyzed by TLC. Fractions containing pure product was pooled, the solvent evaporated obtaining 102.3 mg of 4-bromophenacyl-β-hydroxyisobutyrate. Product was characterized by NMR analysis. HPLC Analysis was done on RP-HPLC) (bridge C18 (5 μm, 4.6×250 mm, Waters) column. Solvent system: A trifluoroacetic acid (TFA) (0.1% v/v) in water, solvent B (0.095% TFA) in CH₃CH:water 4:1; Gradient elution 10-100% B, 30 min, flow-rate 1 mL min⁻¹, 30° C. and detection at 215 nm. The retention time of 4-bromophenacyl-β-hydroxyisobutyrate was 21 min.

### Example 7

### Aldol Addition of Ethanal to 2-Chloroethanal

### Example 7.1

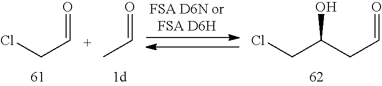

The volume of the reaction was 300 μL but formally it can be scaled up to 5-50 mL (or other amount if needed). 2-Chloroethanal (100 mM) and acetaldehyde (100 mM) were dissolved in 50 mM triethanolamine buffer pH 7.5 (the amount depending on the reaction volume). The reaction was started by the addition of FSA D6N (3 mg mL⁻¹). The reaction mixture was shacked (1000 rpm) at 25° C. After 24 h another extra addition of FSA D6N (3 mg mL⁻¹) can be done if the reaction is not complete (max conversion around 80-85%). When the reaction is completed MeOH (1× reaction volume) is added. The resulting mixture was filtered over activated charcoal. The filtrate was evaporated under vacuum, dissolved in MeOH and adsorbed onto silica. The product adsorbed onto silica was loaded on a column chromatography (3 cm diameter) packed with silica (15 cm of silica bed), sample was eluted with hexane:ethyl acetate mixture in a step gradient manner: 100:0 (100 mL), 70:30 (200 mL), 50:50 (400 mL) and 30:70 (400 mL). The product appeared around the 50:50 hexane:ethyl acetate mixture. Product was compared with an authentic sample obtained using DERA catalyst.

HPLC analysis: Samples (5 μL) were withdrawn immediately after adding the enzyme and after 24 hours, and O-benzylhydroxylamine (50 μL, of a 0.13 mM stock solution in pyridine:methanol:water 33:15:2) was added and placed at room temperature for 5 min. After that, methanol (450 μL) was added and subsequently injected (30 μL) to the HPLC system (column: XBridge® C18, 5 μm, 4.6×250 mm column (Waters)). HPLC analysis: solvent A: 0.1% TFA in H₂O; solvent B: 0.095% TFA in CH₃CN:H₂O 80:20, gradient elution from 10 to 100% of B over 30 min.

### Example 8

### Aldol Addition of Propanone to Propanal

### Example 8.1

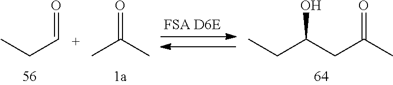

(R)-4-hydroxyhexan-2-one (64): In the reaction vessel lyophilized whole cells of E. coli BL21, previously cultivated for plasmid-instructed expression of FSA D6E (250 mg, equivalent to 15 mg of pure FSA) were suspended in PBS buffer (15 mL, pH 7.0). Acetone (4 mL, 54.5 mmol), freshly distilled propanal (56) (1 mL, 13.9 mmol), and DTT (2 mM) were added. The suspension was shaken on a rotary shaker at 21° C. for 4 days, then centrifuged at 3087×g for 20 min. The supernatant was extracted with diethyl ether (3×20 mL), and the combined organic layers were dried over MgSO₄, filtered, and the solvent evaporated under vacuum. The oily residue containing almost pure product 64 (550 mg, 33.9%) was analyzed by NMR spectroscopy. ¹H NMR (300 MHz, MeOD): δ=1.05 (t, 3H, J (6/5)=7.5 Hz, H-6), 1.58 (m, 2H, H-5), 2.29 (s, 3H, H-1), 2.68 (d, 2H, J (3/4) =6.4 Hz, H-3), 4.06 (tt, 1H, H-4). ¹³C NMR (75 MHz, MeOD): δ=10.24 (C-6), 30.80 (C-1), 30.93 (C-5), 51.23 (C-3), 70.05 (C-4), 210.80 (C-2).

### Example 9

### Aldol Addition of Propanone to n-Butanal

### Example 9.1

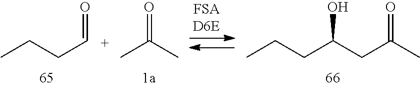

(R)-4-hydroxyheptan-2-one (66): In the reaction vessel, lyophilized whole cells of E. coli BL21, previously cultivated for plasmid-instructed expression of FSA D6E (200 mg, equivalent to 12 mg of pure FSA) were suspended in PBS buffer (15 mL, pH 7.0). Acetone (1a) (4 mL, 54.5 mmol), freshly distilled butanal (65) (1 mL, 11.1 mmol), and DTT (2 mM) were added. The suspension was shaken on a rotary shaker at 21° C. for 4 days, then centrifuged at 3087×g for 20 min. The supernatant was extracted with diethyl ether (3×20 mL), and the combined organic layers were dried over MgSO₄, filtered, and the solvent evaporated under vacuum. The oily residue containing almost pure product 66 (141 mg, 9.7%) was analyzed by NMR spectroscopy. ¹H NMR (300 MHz, MeOD): δ=0.99 (t, 3H, J (7/6)=7.1 Hz, H-7), 1.47 (m, 4H, H-5,6), 2.22 (s, 3H, H-1), 2.61 (d, 2H, J (3/4)=6.1 Hz, H-3), 4.08 (tt, 1H, H-4). ¹³C NMR (75 MHz, MeOD): δ=14.33 (C-7), 19.75 (C-6), 30.71 (C-1), 40.60 (C-5), 51.86 (C-3), 68.60 (C-4), 210.85 (C-2).

### Example 10

### Aldol Addition of Propanone to n-Pentanal

### Example 10.1

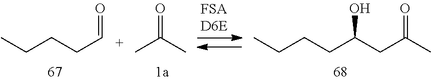

(R)-4-hydroxyoctan-2-one (68): In the reaction vessel, lyophilized whole cells of E. coli BL21, previously cultivated for plasmid-instructed expression of FSA D6E (250 mg, equivalent to 15 mg of pure FSA) were suspended in PBS buffer (15 mL, pH 7.0). Acetone (1a) (4 mL, 54.5 mmol) and freshly distilled pentanal (67) (1 mL, 9.4 mmol) were added. The suspension was shaken on a rotary shaker at 21° C. for 6 days, then centrifuged at 3087×g for 20 min. The supernatant was extracted with diethyl ether (3×20 mL), and the combined organic layers were dried over MgSO₄, filtered, and the solvent evaporated under vacuum. The oily residue containing almost pure product 68 (250 mg, 18.4%) was analyzed by NMR spectroscopy. ¹H NMR (300 MHz, CDCl₃): δ=0.81 (t, 3H, J=6.4 Hz, H-8), 1.15-1.48 (m, 6H, H-5, 6, 7), 2.09 (s, 3H, H-1), 2.50 (m, 2H, H-3), 3.95 (tt, 1H, J=7.4/3.4 Hz, H-4). ¹³C NMR (75 MHz, CDCl₃): δ=13.95 (C-8), 22.56 (C-7), 27.60 (C-6), 30.69 (C-1), 36.20 (C-5), 50.08 (C-3), 67.57 (C-4), 209.89 (C-2).

### Example 11

### Aldol Addition of Propanone to Isopentanal

### Example 11.1

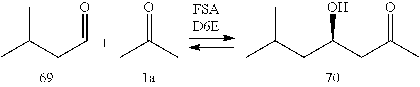

(R)-4-hydroxy-6-methylheptan-2-one (70): In the reaction vessel lyophilized whole cells of E. coli BL21, previously cultivated for plasm id-instructed expression of FSA D6E (191 mg, equivalent to 11 mg of pure FSA) were suspended in PBS buffer (15 mL, pH 7.0). Acetone (1a) (4 mL, 54.5 mmol), freshly distilled isopentanal (69) (1 mL, 9.3 mmol), and DTT (2 mM) were added. The suspension was shaken on a rotary shaker at 21° C. for 4 days, then centrifuged at 3087×g for 20 min. The supernatant was extracted with diethyl ether (3×20 mL), and the combined organic layers were dried over MgSO₄, filtered, and the solvent evaporated under vacuum. The oily residue containing almost pure product 70 (284 mg, 21.2%) was analyzed by NMR spectroscopy. ¹H NMR (300 MHz, CDCl₃): δ=0.87 (d, 6H, J=6.6 Hz, H-7/1′), 1.09 (m, 1H, J=13.7 Hz, H-6), 1.42 (m, 1H, J=13.7 Hz, H-5a), 1.74 (m, 1H, H-5b), 2.13 (s, 3H, H-1), 2.52 (m, 2H, H-3), 4.08 (m, 1H, J=4.4 Hz, H-4). ¹³C NMR (75 MHz, CDCl₃): δ=22.06 (C-6), 23.35 (C-7/1′), 24.39 (C-7/1′), 30.84 (C-1), 45.58 (C-5), 50.57 (C-3), 65.69 (C-4), 210.15 (C-2).

### Example 12

### Aldol Addition of Propanal to Propanal

### Example 12.1

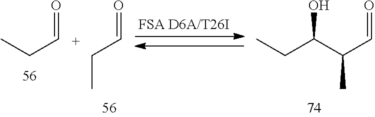

(2S,3R)-3-hydroxy-2-methylpentanal (74). The enzyme catalyst FSA D6A/T26I (35 mg) was dissolved in triethanolamine buffer (100 mL; 50 mM, pH 7.4) containing DTT (2 mM). After addition of freshly distilled propanal (1500 μL, 20.9 mmol) the vessel was stoppered and reaction mixture stirred at 20° C. for 3 days. Then CaCl₂ solution was added (1 mM final concentration) followed by proteinase K (51 U), and the pH adjusted to 7.0 using satd aq NaHCO₃ solution. This mixture was incubated at 55° C. for 2 h during which the turbid solution became clear. The product was extracted with ethyl acetate (4×60 mL) with TLC control. The combined organic layers were dried (MgSO₄), filtered, and the solvent evaporated at 40° C. under vacuum. The oily residue was purified by silica column chromatography using cyclohexane-ethyl acetate (3:1) as eluent to give pure product (230 mg, 9.5%). ¹H NMR (300 MHz, CDl₃): δ=0.79 (d, 3H, J=7.05 Hz, H-5), 0.84 (t, 3H, J=7.45 Hz, H-2′), 1.35 (m, 2H, H-4), 1.65 (m, 1H, H-2), 3.50 (m ,2H, H-3), 3.58 (m, 1H, H-1), 3.96 (brs, 2H, OH-1′, OH-3′). ¹³C NMR (75 MHz, CDCl₃): δ=9.90 (C-5), 10.57 (C-2), 26.71 (C-4), 38.75 (C-2), 66.19 (C-1), 74.96 (C-3).

### Reduction of Aldehyde 74 and Diol Protection for Configurational Assignment.

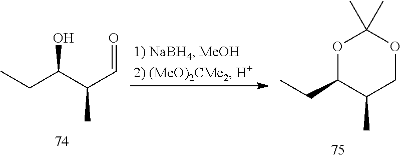

(4R,5R)-4-Ethyl-2,2,5-trimethyl-1,3-dioxane (75). The aldehyde 74 (1 eq) was dissolved in MeOH (1.4 mL per mmol aldehyde) and treated portion wise with NaBH₄ (1.5 eq per mmol) with stirring at room temperature. After 2 h, 2 volumes of brine was added and a solid precipitated. The mixture was slowly treated with deionisized water until the turbidity disappeared (ca. 4 m L/mL MeOH). The solution was extracted with ethyl acetate (6×20 mL; TLC control). The combined organic phases were dried (MgSO₄), filtered and concentrated under vacuum at 40° C. The residue (130 mg, 55.5%) was used directly in the next step without further purification.

The diol was taken up in dry acetone (2.5 mL) followed by addition of dimethoxypropane (2.5 mL). To this was added molecular sieves (250 mg; 3 Å) and a catalytic quantity of p-TosOH. The vessel was closed by using a septum and stirred at 20° C. for 2.5 h. After addition of satd NaHCO₃ solution (2.5 mL) the mixture was extracted using diethyl ether (3×5 mL). The combined organic phases were dried (MgSO₄), filtered and concentrated under vacuum to furnish the acetal 75 (300 mg, 29.9%). ¹H NMR (300 MHz, CDCl₃, 30% C₆D₆): δ=1.54 (t, 3H, J (4′,3′)=7.4 Hz, H-4′), 1.73 (d, 3H, J (5′,5)=6.8 Hz, H-5′), 1.83 (m, 1H, H-5), 1.95 (ddq, 1H, J (3′,4′)=7.4 Hz, H-3′a), 2.03 (s, 3H, H-1′), 2.14 (s, 3H, H-2), 2.20 (ddt, 1H, J (3′,4′)=7.4 Hz, H-3′b), 4.16 (dd, J (6b,5)=1.66, J (6a,6b)=11.40 Hz, 1H, H-6a), 4.32 (ddd, 1H, J=2.5, 5.9, 8.1 Hz, H-4), 4.56 (dd, J (6a,5)=2.8, J (6b,6a)=11.40 Hz, 1H, H-6b) ppm. ¹³C NMR (75 MHz, CDCl₃ 30% C₆D₆): δ=9.56 (C-5′), 10.33 (C-4′), 18.87 (C-1′), 29.81 (C-3′), 29.80 (C-2′), 31.31 (C-5), 66.65 (C-6), 72.82 (C-4), 98.22 (C-2) ppm.

### Example 13

### Aldol Addition of Butanal to Butanal

### Example 13.1

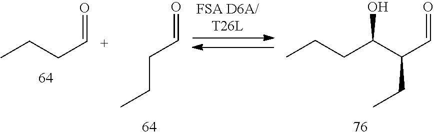

(2S,3R)-2-ethyl-3-hydroxyhexanal (76): The enzyme catalyst FSA D6A/T26L (35 mg) was dissolved in triethanolamine buffer (100 mL; 50 mM, pH 7.4) containing DTT (2 mM). After addition of freshly distilled butanal (64) (400 μL, 20.9 mmol) the vessel was stoppered and reaction mixture stirred at 20° C. for 3 days. Then CaCl₂ solution was added (1 mM final concentration) followed by proteinase K (51 U), and the pH adjusted to 7.0 using satd aq NaHCO₃ solution. This mixture was incubated at 55° C. for 2 h during which the turbid solution became clear. The product was extracted using ethyl acetate (4×60 m L) with TLC control. The combined organic layers were dried (MgSO₄), filtered, and the solvent evaporated at 40° C. under vacuum. The oily residue was purified by silica column chromatography using cyclohexane-ethyl acetate (3:1) as eluent to give pure product 76 (90 mg, 26.9%). ¹H NMR (300 MHz, CHCl₃): δ=1.31 (t, 6H, J=7.5 Hz, H-5′, H-T), 1.36 (m, 1H, J=10.5 Hz, H-5), 1.66 (m, 2H, H-4a′, H-6a), 1.72-1.85 (m, 2H, H-4b′, H-6b), 1.77 (s, 3H, H-1′), 1.78 (s, 3H, H-2′), 1.90 (m, 1H, H-3a), 2.12 (m, 1H, J=3.3 Hz, H-3b), 4.17 (dd, 1H, J (6a,5)=1.8 J (6a,6b)=11.8 Hz, H-6a), 4.24 (m, 2H, H-4, H-6b) ppm. ¹³C NMR (75 MHz, CHCl₃): δ=12.06 (C-7′), 14.06 (C-5′), 16.15 (C-4′), 18.83 (C-6′), 19.15 (C-1′), 29.79 (C-2′), 34.88 (C-3′), 39.11 (C-5), 62.63 (C-6), 71.94 (C-4), 98.44 (C-2) ppm.

### Reduction of Aldehyde 76 and Diol Protection for Configurational Assignment.

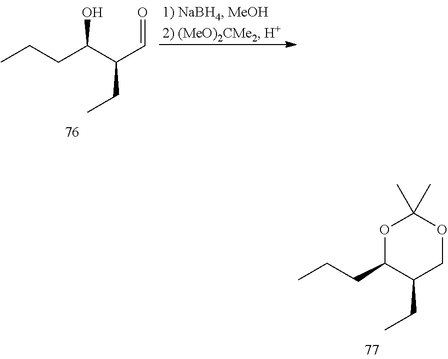

(4R,5R)-5-Ethyl-2,2-dimethyl-4-propyl-1,3-dioxane (77). The aldehyde 76 (1 eq) was dissolved in MeOH (1.4 mL per mmol aldehyde) and treated portion wise with NaBH₄ (1.5 eq per mmol) with stirring at room temperature. Work-up and acetal protection using dim ethoxypropane in acetone with acid catalysis was essentially performed as for 75. Extraction of product 77 was performed using cyclohexane, and purification was achieved by silica gel column chromatography (ethyl acetate-cyclohexane 1:10). ¹H NMR (300 MHz, CDCl₃, 30% C₆D₆): δ=1.54 (t, 3H, J (4′,3′)=7,4 Hz, H-4′), 1.73 (d, 3H, J (5′,5)=6.8 Hz, H-5′), 1.83 (m, 1H, H-5), 1.95 (ddq, 1H, J (3′,4′)=7.4 Hz, H-3′a), 2.03 (s, 3H, H-1′), 2.14 (s, 3H, H-2′), 2.20 (ddt, 1H, J (3′,4′)=7.4 Hz, H-3′b), 4.16 (dd, J (6b,5)=1.66, J (6a,6b)=11.40 Hz, 1H, H-6a), 4.32 (ddd, 1H, J=2.5, 5.9, 8.1 Hz, H-4), 4.56 (dd, J (6a,5)=2.8, J (6b,6a)=11.40 Hz, 1H, H-6b) ppm. ¹³C NMR (300 MHz, CDCl₃ 3 30% C₆D₆): δ=9.56 (C-5′), 10.33 (C-4′), 18.87 (C-1), 29.81 (C-3′), 29,80 (C-2′), 31.31 (C-5), 66.65 (C-6), 72.82 (C-4), 98.22 (C-2) ppm.

1. A fructose-6-phosphate aldolase variant comprising:
a. an amino acid sequence having at least 80%, preferably at least 90%, more preferably at least 99%, sequence identity with SEQ ID NO: 1, and
b. an amino acid substitution at position 6, corresponding to positions 1 to 220 of SEQ ID NO: 1.

2. The variant according to claim 1, wherein the amino acid substitution is by an amino acid selected from the list consisting of: A, L, N, Q, S, T, E, H, V, G, I or P, preferably by H, N, Q, L, T, E or A.

3. The variant according to any of claim 1 or 2, wherein said variant further comprises an amino acid substitution at position 26, corresponding to positions 1 to 220 of SEQ ID NO: 1.

4. The variant according to claim 3, wherein the amino acid substitution at position 26 is by an amino acid selected from the list consisting of: V, A, L, I or P, preferably by L or I.

5. The variant according to any of claim 1 or 2, wherein said variant further comprises an amino acid substitution at position 165, corresponding to positions 1 to 220 of SEQ ID NO: 1, by the amino acid G.

6. The variant according to any one of claims 1 to 5, wherein said variant comprises the amino acid substitution D6N or D6H.

7. A nucleic acid sequence encoding the variant according to any one of claims 1 to 6.

8. A gene construct comprising the nucleic acid sequence according to claim 7.

9. A host cell comprising the nucleic acid sequence according to claim 7 or the gene construct according to claim 8.

10. Use of the host cell according to claim 9 for producing the variant according to any one of claims 1 to 6.

11. Use of the variant according to any one of claims 1 to 6 or the host cell according to claim 9 as a biocatalyst for aldol carboligation reactions.

12. Use according to claim 11, wherein the aldol carboligation reaction takes place between two aldehydes or between an aldehyde and a ketone, preferably wherein the aldehydes are selected from the list consisting of: 2-chloroethanal, formaldehyde, benzyloxyethanal, propanal, pentanal, isopentanal, ethanal, butanal, 3-hydroxypropanal, 3-hydroxybutanal, N-Cbz-3-am nopropanal or N-Cbz-glycinal, and the ketone is selected from the list consisting of: propanone, butanone, cyclobutanone, cyclopentanone or 3-pentanone.

13. A method for aldol carboligation comprising the following steps:
a. reacting two aldehydes or an aldehyde and a ketone in the presence of the variant according to any one of claims 1 to 6 or the host cell according to claim 9, and
b. recovering the product of the reaction performed in step (a).

14. The method according to claim 13, wherein the aldehydes are selected from the list consisting of: 2-chloroethanal, formaldehyde, benzyloxyethanal, propanal, pentanal, isopentanal, ethanal, butanal, 3-hydroxypropanal, 3-hydroxybutanal, N-Cbz-3-aminopropanal or N-Cbz-glycinal, and the ketone is selected from the list consisting of: propanone, butanone, cyclobutanone, cyclopentanone or 3-pentanone.
